(19) United States
(12) Patent Application Publication
(10) Pub. No.: US 2005/0089524 A1
(43) Pub. Date: Apr. 28, 2005

(54) Compositions and compounds for use as molecular adjuvant for a nicotine vaccine

(76) Inventors: Sam D.G. Sanderson, Omaha, NE (US); Jonathan L. Vennerstrom, Omaha, NE (US); Geoffrey M. Thiele, Omaha, NE (US); Maniyan Parameswaran, Woburn, MA (US); Rick A. Bevins, Lincoln, NE (US); Cheruku R. Srinivasa, Woburn, MA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/506,094

(22) PCT Filed: Feb. 20, 2003
(86) PCT No.: PCT/US03/05015

Related U.S. Application Data
(60) Provisional application No. 60/360,967, filed on Mar. 1, 2002.

(51) Int. Cl. A61K 39/00; C07K 14/47

(52) U.S. Cl. 424/185.1; 530/409

(57) ABSTRACT

Compounds are disclosed comprising molecular adjuvants having an antigen presenting cell-targeting ligand linked to a nicotine hapten. Methods are disclosed for employing the compounds as a nicotine vaccine for treatment or prevention of nicotine addiction.

Pursuant to 35 U.S.C. §202(c), it is hereby acknowledged that the U.S. Government has certain rights in the invention described herein, which was made, in part, with funds from the National Institutes of Health, grant numbers CA 57362 and CA 36727.

### FIELD OF THE INVENTION

The present invention relates to the field of vaccines and stimulation of acquired immunity. In particular, the present invention provides novel compositions for use as a nicotine vaccine.

### BACKGROUND OF THE INVENTION

The disease consequences of habitual tobacco smoking are irrefutable. Smoking tobacco is a well-established causative factor in the pathogenesis of cancers of the lung, oral cavity, nasal/sinus cavities, esophagus, and bladder. Likewise, inhalation of tobacco smoke is a major etiologic factor in the expression of airway inflammatory disorders such as chronic obstructive pulmonary disease and allergic asthma. Smoking is a well-known risk factor for developing coronary heart disease and stroke. In addition, cigarette smoking contributes to an increase in systemic bone loss and an impairment in the ability to heal bone grafts and fractures. Smokers have less strong, less healthy, mineral-deficient bones with reduced bone blood supply and fewer and less functional bone-forming cells compared to their non-smoking counterparts. Bone loss in the oral cavity associated with cigarette smoking is well documented in the dental literature. Cigarette smoking is a major risk factor for periodontitis and has been directly linked to alveolar bone loss, overall tooth loss, and an impaired healing/fusion of dental implants.

The adverse health effects associated with tobacco smoking also extend to those exposed to secondary smoke. Chronic exposure to secondary smoke is a growing public health problem, which has been directly linked to an increased incidence of sudden infant death syndrome in infants of mothers who smoke, low birth weight of infants from mothers who smoked during pregnancy, middle ear infections in children, exacerbation of childhood and adult asthma, bronchitis, lung/nasal cancers, and ischemic heart disease in children and adults.

As well established as the adverse health consequences of smoking, so too are the health benefits derived from smoking cessation. Former smokers, for example, are at a significantly lower risk for total mortality in all smoking-related disease categories relative to their smoking counter-parts. Moreover, the risk for total mortality among all disease categories for those who quite smoking and maintain compliance eventually reaches a level comparable to those who never smoked.

Quitting smoking, however, can be difficult and even with support, only a third of the smoking population is able to quit. Since tobacco dependence results from an addiction to nicotine, strategies aimed at smoking cessation target nicotine's addictive effects. The mainstay of these treatments is nicotine replacement therapy with gradually decreasing doses of nicotine delivered via transdermal patches, gum, or inhaled nicotine formulations. Although this approach has proven beneficial to some individuals, the continued administration of the principal addictive substance in cigarette smoke is not an ideal approach to smoking cessation and the nicotine itself may exert adverse cardiovascular effects. Nicotine addiction is also treated with neuroreceptor antagonists/partial agonists to modulate neurotransmitter pathways in the brain. Since these receptors and their signaling pathways also mediate normal brain function, side-effects are common with the use of these drugs. Thus, there is a growing need for the development of improved strategies to help individuals quit smoking and maintain long-term compliance in an effort to prevent the costly and debilitating diseases caused by smoking.

One innovative approach to smoking cessation and long-term compliance is to treat the addictive properties of nicotine via immunization with a nicotine vaccine. The rationale to this approach is that nicotine-specific antibodies generated in response to the vaccine bind circulating nicotine outside of the central nervous system and reduce drug access to receptor sites in the brain. This peripheral site of action, along with the high specificity and affinity of nicotine antibodies, makes vaccination an attractive therapeutic approach to smoking cessation. Also, the use of a nicotine vaccine has the potential of inducing a “memory” immune response, wherein anti-nicotine immunity may be invoked when one in exposed to nicotine, an attractive feature for ensuring long-term compliance. Moreover, a nicotine vaccine would be significantly less expensive than conventional treatments for nicotine addiction and smoking cessation.

Toward that end, drug-specific vaccines have been used successfully to attenuate the behavioral/psychoactive effects of cocaine (Fox et al., (1996) Nature Med. 2:1129-1132), heroin (Killian et al., (1978) Pharmacol. Biochem. Behav. 9:34-352), and nicotine (Hieda et al., (1997) J. Pharmacol. Exp. Therap. 283:1076-1081; and Pentel et al., (2000) Pharmacol. Biochem. Behav. 65:191-198) in rodent models. The success of these applications emanated from the vaccine's ability to induce drug-specific antibodies that bound the drug and limited its distribution to the brain where the addictive effects are exerted. These anti-drug vaccines were composed of the drug conjugated to a large carrier protein and immunizations were performed in the presence of added adjuvant. The two nicotine vaccines, for example, were made by conjugating 6-(carboxymethylureido)-(±)-nicotine to keyhole limpet hemocyanin (KLH) (Hieda et al., (1997) J. Pharmacol. Exp. Therap. 283:1076-1081) and trans-3′-aminomethylnicotine to Pseudomonas aeruginosa expoprotein A (rEPA) (Pentel et al., (2000) Pharmacol. Biochem. Behav. 65:191-198). Immunizations were performed in rabbits and rats in the presence of complete and incomplete Freund's adjuvant. Under these conditions, both nicotine vaccines were capable of inducing nicotine-specific antibodies. These antibodies, when used in passive immunizations protocols, were shown to bind plasma nicotine in rats, which minimized its adverse cardiovascular effects, and decreased nicotine distribution to the brain, which attenuated its psychoactive/behavioral effects. Also, when used in active immunization protocols, both vaccines generated nicotine-specific antibodies that were capable of altering the plasma and brain concentrations of nicotine in rats.

While the results detailed above illustrate the therapeutic potential of a nicotine vaccine for controlling the addictive properties of nicotine, these approaches do not provide a viable strategy for smoking cessation and long term compliance. The nicotine-carrier protein designs used in these studies, for example, are limited by their inability to direct the nicotine antigen to and activate antigen presenting cells (APC), which are essential in processing and presenting the antigen to helper T cells. Such APC-mediated engagement of helper T cells is critical for the T helper cells to release the cytokines necessary to “help” the antibody-producing B cells generate the antigen-specific antibodies. A second limitation to these vaccine designs is their reliance on the use of harsh adjuvants (such as Freund's) to compensate for this lack of APC specificity. These adjuvants comprise oily solutions containing components, such as lipopolysaccharides that stimulate generalized immune responses. A third limitation is the lack of control in attaching the nicotine hapten to the carrier protein in a consistent and reproducible manner. Attachment of a small hapten like nicotine to a large carrier protein with many potential reactive sites allows for the possibility of hapten-to-carrier ratios varying from lot to lot, causing variation in vaccine efficacy. Also, the immunogenic character of the carrier protein may be altered or diminished by the random attachment of large numbers of haptens to immunologically important sites.

Accordingly, a need exists for a nicotine vaccine that is capable of causing smoking cessation and long term compliance. The present invention addresses this need by providing a composition for use as a nicotine vaccine comprising a molecular adjuvant that is able to deliver both stimulatory signals and the nicotine antigen to APCs, thereby inducing an anti-nicotine antibody response with little or no inflammatory side-effects and without reliance on other added adjuvants.

Several publications are referenced in this application by numerals in parenthesis in order to more fully describe the state of the art to which this invention pertains. Full citations for these references are found at the end of the specification. The disclosure of each of these publications is incorporated by reference herein.

### SUMMARY OF THE INVENTION

The present invention provides novel compositions and methods for delivering specific antigens to antigen-presenting cells, and simultaneously delivering signals to those cells that produce a desired immune response. The compositions of the invention are sometimes referred to herein as “APC-targeted activating antigens.”

According to one aspect of the invention, these APC-targeted activating antigens, which elicit an immune response mediated by an antigen-presenting cell, comprise at least one antigenic moiety functionally linked to at least one targeting moiety that binds specifically to a characteristic determinant on the antigen-presenting cell. For purposes of the present invention, the term “functionally linked” is defined generally as linking of the moieties in such a way that each moiety retains its intended function. This is particularly relevant with respect to the targeting moiety, which is designed to bind to a characteristic determinant on the antigen-presenting cell.

Antigen-presenting cells contemplated for targeting according to the present invention include, but are not limited to, monocytes, dendritic cells, macrophages, B cells and some T cells. In preferred embodiments of the invention, the characteristic determinant on the selected APC is a cell surface receptor and the targeting moiety of the APC-targeted antigen is a ligand that binds to the receptor. It is particularly preferred that the cell surface receptor be an immunomodulatory receptor. Suitable cell surface receptors include, but are not limited to, C5a receptor, IFNγ receptor, CD21 (C3d) receptor, CD64 (FcγRI) receptor, and CD23 (FcγRII) receptor.

One exemplary APC-targeted antigen of the invention is designed to bind to the C5a receptor, and the targeting moiety is a C5a receptor ligand, which is preferably a peptide analog of C5a corresponding to the C-terminal 10 residues of C5a. Another exemplary composition of the present invention is designed to bind to the IFNγ receptor, and comprises a targeting moiety which is a IFNγ receptor ligand, preferably a peptide analog of IFNγ corresponding to the N-terminal 39 residues of IFNγ.

The antigenic moiety of the APC-targeted antigens of the invention can comprise essentially any antigenic substance, including, but not limited to, peptides and proteins, glycopeptides and glycoproteins, phosphopeptides and phosphoproteins, lipopeptides and lipoproteins, carbohydrates, nucleic acids and lipids. The APC-targeted antigens can comprise more than one antigenic moiety, and likewise can comprise more than one targeting moiety. Moreover, these moieties can be functionally linked in several fashions. For instance, if “T” represents a targeting moiety, and “Ag” represents an antigenic moiety, the APC-targeted antigens of the present invention may be oriented as follows:
Ag-T;
T-Ag;
T₁-Ag-T₂;
T₁-[Ag]ₙ-T₂ (wherein [Ag]ₙ represents a multiplicity of antigens.

Examples of the general formulas set forth above include:
Ag-C5a agonist peptide;
IFNγ peptide-Ag;
IFNγ peptide-[Ag]ₙ-C5a agonist peptide.

According to other aspects of the present invention, methods are provided for using the APC-targeted antigens of the invention. These include methods of activating an antigen-presenting cell with a targeting ligand and methods of eliciting an antigen presenting cell-mediated immune response in a subject in which such a response is desired. In particular, a method is provided to treat or prevent smoking addiction by administration of a therapeutic amount of compositions of the invention. General methods of immunizing or vaccinating a patient requiring such treatment, methods of treating a tumor, and methods for producing antibodies specific for a pre-determined antigen for use as research tools or for diagnostic purposes are also contemplated to be within the scope of the present invention.

The numerous features and advantages of the compositions and methods of the present invention are described more fully in the detailed description set forth below.

### BRIEF DESCRIPTION OF THE DRAWINGS

FIG. 1 is a graph illustrating the antibody titer produced in mice immunized with the indicated peptide constructs, as determined by radioimmunoassay, and shows the relationship between the amount of ¹²⁵I-goat anti-mouse antibody bound vs. the dilution factor of mouse sera which had been incubated in microtiter wells coated with the MUC1 epitope peptide.

FIG. 2 is a graph illustrating the increase in antibody titer in the sera of mice collected either before (pre) or after immunization with peptides 3 (YKQGGFLGLYSFKPMPLaR) (SEQ ID NO:2) and 4 (YSFKPMPLaRKQGGFLGL) (SEQ ID NO:5) as determined by radioimmunoassay and shows the relationship between the amount of ¹²⁵I-goat anti-mouse antibody bound and the dilution factor of mouse sera which had been incubated in microtiter wells coated with MUC1 epitope peptide. Note that peptides 3 and 4 comprise two moieties, a targeting ligand and an antigen to which an immune response is desired.

FIG. 3 is a graph illustrating the titers of antibody classes and subclasses produced in mice following immunization with peptide 3 (YKQGGFLGLYSFKPMPLaR) (SEQ ID NO:2) as determined by ELISA using rabbit anti-mouse IgA, IgG1, IgG2a, IgG2b, IgG3, and IgM, followed by goat anti-rabbit conjugated to peroxidase and detected using p-nitrophenyl phosphate cleavage monitored at 405 nm.

FIG. 4 is a graph illustrating the specificity of binding of the antibody subclasses in sera from mice immunized with peptide 3 (YKQGGFLGLYSFKPMPLaR) (SEQ ID NO:2) as determined by ELISA using binding to microtiter wells coated with MUC1 epitope peptide and detection with rabbit anti-mouse IgG2a, IgG2b, or IgM followed by incubation with goat anti-rabbit conjugated to peroxidase and detected using p-nitrophenyl phosphate cleavage monitored at 405 nm.

FIG. 5 is a graph showing the HBsAg-specific CTL response is induced only by C5-a-active constructs. BALB/c mice received three s.c. injections at 21 day intervals using 50 μg doses of the HBsAg-L^{d} MHC class I restricted peptide (S₂₈₃₉) synthesized with two Arg residues appended to the C-terminal end (IPQSLDSWWTSLRR; SEQ ID NO: 18), the double-Arg-linked C5a-active construct (IPQSLDSWWTSLRRYSFKPMPLaR; SEQ ID NO: 14) and the double-Arg-linked C5a-inactive constructs (IPQSLDSWWTSLRRYSFKPMPLaRG; SEQ ID NO: 17 and YSFKPMPLaRRRIPQSLDSWWTSL; SEQ ID NO: 16). Pooled spleen cell suspensions were prepared from two mice in each group 14 days following the third injection. The cell suspensions were cultured in the presence of the S₂₈₃₉ peptide (75 nM) for four days and used as effector cells against ⁵¹Cr-labeled P815S (HBsAg transfectant) or P815 targets. Percent specific lysis of ⁵¹Cr labeled P815S cells at various effector to target ratios is shown and represent the means of triplicate determinations. Lysis of ⁵¹Cr labeled P815 cells at an effector-to-target ratio of 50:1 was less than 5% (not shown). The data are representative of three separate experiments.

FIG. 6 is a graph showing that HBsAg-specific CTL responses are induced only by the C5a-active, protease-sensitive-linked constructs. BALB/c mice received three s.c. injections at 21 day intervals using 50 μg doses of the C5a-active constructs in which the HBsAg-L^{d} MHC class I restricted peptide (S₂₈-39) was covalently attached directly to the N-terminus of the C5a agonist (IPQSLDSWWTSLYSFKPMPLaR; SEQ ID NO: 13), spaced by two Arg residues (IPQSLDSWWTSLRRYSFKPMPLaR; SEQ ID NO: 14), or spaced by the furin protease-sensitive sequences RVRR (SEQ ID NO: 19) (IPQSLDSWWTSLRVRRYSFKPMPLaR; SEQ ID NO: 15). Spleen cell suspensions were prepared from two mice in each group 14 days following the third injection. The cell suspensions were cultured in the presence of the S₂₈₋₃₉ peptide (75 nM) for four days and used as effector cells in ⁵¹Cr-release assays against P815S or P815 targets. Percent specific lysis of ⁵¹Cr-labeled P815S is shown and represent the means of triplicate determinations. Lysis of ⁵¹Cr-labeled P815 cells by these effector cells was less than 5% at an effector-to-target ratio of 50:1 (not shown).

FIG. 7 is a graph illustrating the antibody reactivity of the indicated nicotine vaccine construct.

FIG. 8, Panel A depicts the total number of dipper entries for each treatment regime employed, as indicated and Panel B depicts the index of learning-evaluation score for the first light (before sucrose is given) for each treatment regime employed, as indicated.

FIGS. 9 and 10 collectively depict the ability of the nicotine vaccine to generate nicotine specific antibodies in rats immunized with the vaccine.

### DETAILED DESCRIPTION OF THE INVENTION

A major obstacle in the development of vaccines and other immunostimulatory agents is the inability of some antigens to be readily taken up and processed by antigen presenting cells. Uptake of antigens by APCs is an essential step for stimulating an effective immune response, since the immune system recognizes the antigen only after it has been processed by the APC and presented on the surface of the APC in conjunction with the major histocompatibility complex (MHC).

It is known that APCs, including dendritic cells, monocytes, macrophages and B cells, possess functional receptors for numerous molecules that modulate the immune response. It has now been discovered in accordance with the present invention that ligands which bind to these receptors can be conjugated to weakly immunogenic antigens for example, as a way of delivering antigens to the antigen presenting pathway of the APC and simultaneously activating the antigen presenting capacity of the APC. Thus, these conjugates bind to a receptor on the APC surface, transduce a biological signal, and are internalized by the APC. The antigenic moiety of the conjugate is thereby delivered to the antigen presenting pathway of the APC along with the simultaneous activation of the APC.

The above-described conjugates are sometimes referred to herein as “molecular adjuvants” or “APC-targeted activating antigens.” The APC-targeted activating antigens of the invention are designed to elicit immune responses mediated by one or more types of antigen presenting cells. Accordingly, an APC-targeted activating antigen comprises at least one antigenic moiety linked to a targeting and activating moiety that binds specifically to at least one characteristic determinant on the selected antigen presenting cell type. This binding is followed by internalization of the APC-targeted antigen and results in presentation of the antigen moiety on the surface of the APC. For purposes of the present invention, the term “antigenic moiety” may refer to any substance to which it is desired that an immune response be produced. The selected antigenic moiety may or may not be capable of eliciting an immune response by conventional means.

The term “determinant” is used herein in its broad sense to denote an element that identifies or determines the nature of something. When used in reference to an antigen presenting cell, “determinant” means that site on the antigen presenting cell which is involved in specific binding by the targeting ligand moiety of the molecular adjuvant of the invention.

The expression “characteristic determinant” as used herein, signifies an epitope (or group of epitopes) that serves to identify a particular population of antigen presenting cells and distinguish it from other antigen presenting cell populations. Cell-associated determinants include, for example, components of the cell membrane, such as membrane-bound proteins or glycoproteins, including cell surface antigens, histo-compatibility antigens or membrane receptors.

The expression “specific binding”, as used herein refers to the interaction between the targeting ligand moiety and a characteristic determinant on the antigen presenting cell population sought to be activated in accordance with this invention, to the substantial exclusion of determinants present on other cells.

Certain exemplary compositions of the invention have been synthesized, and have been shown to elicit APC-mediated immune responses in accordance with the mechanisms described above. For example, antigenic epitopes have been conjugated to the amino-terminal end of a C5a decapeptide agonist capable of binding to C5a receptors present on the surface of many APCs. Mice that were inoculated with an epitope of human MUC1 (a cell surface-associated mucin) conjugated to such a C5a agonist exhibited pronounced antibody titers against the MUC1 epitope, including high titers of specific antibodies with isotypes IgG2a and IgG2b. Mice that were inoculated with (1) MUC1 epitope alone, (2) C5a agonist alone, (3) unconjugated MUC1 epitope and C5a agonist together, or (4) C5a agonist conjugated to MUC1 epitope in a manner in which the biological activity of the C5a moiety was blocked, did not express a significant specific immune response. These results are described in greater detail in Example 1. Similar results were observed with conjugates of C5a agonist to a 12 kDa polypeptide, serum amyloid A (SAA), as described in greater detail in Example 2. In addition, Example 6 depicts the results of the C5a agonist conjugated to various nicotine constructs. These data tend to demonstrate the feasibility of the invention, which is to use receptor-binding ligands as a way to deliver antigens to APCs, with the simultaneous activation of APCs by the ligand moiety.

As described in greater detail below, the C5a receptor is only one of many receptors expressed on APCs. This invention encompasses the use of various ligands with selectivity to other receptors that mediate signal transduction events in the APCs, to be used alone or in conjunction with C5a agonists to influence the nature of immune response generated, i.e., humoral, cellular, Th1, Th2, and the like. Vaccines and other immunotherapeutic agents can be prepared with an array of such targeting moieties that serve to target the antigen moiety to a specific population of APCs and simultaneously activate these and other cells involved in various immune modulatory pathways.

The detailed description below sets forth preferred embodiments for making and using the targeted antigens of the present invention. To the extent that specific compounds and reagents are mentioned, these are for the purposes of illustration, and are not intended to limit the invention. Any biochemical, molecular or recombinant DNA techniques not specifically described are carried out by standard methods, as generally set forth for example, in Ausubel et al., “Current Protocols in Molecular Biology,” John Wiley & Sons, Inc., 1995.

I. Preparing APC-Targeted Activating Antigens

A. Selection of Components

Antigen presenting cells have various receptors on their surfaces for known ligands. Binding of ligands to these receptors results in signal transduction events that stimulate immune or tolerance responses. Many of these receptors are known to internalize and recycle in the cell. Others are suspected of doing the same. As such, these receptors are ideal targets for delivering antigens and activation signals simultaneously to APCs.

As discussed above, APCs include several cell types including macrophages, monocytes, dendritic cells, B cells, some T cells and other poorly characterized cell types. It is believed that these different classes of APCs can produce different types of immune responses. Accordingly, by targeting a receptor prevalent on a specific population of APCs, a particular desired immune response may be favored. An exemplary list of receptors contemplated for targeting in the present invention, and the rationale for their selection, is set forth below. These APC receptors are particularly appropriate for use in the present invention based on the following criteria: they are receptors expressed on APCS; the receptors are internalized upon binding of ligand; the receptors can transmit signals in the cells that influence antigen processing and presentation by these cells; some of the receptors are believed to be involved in signaling Th1 type cellular responses, whereas others are predicted to generate Th2 type humoral responses. The list set forth below is not exhaustive, but merely representative of the type of targeted receptors preferred in practicing the present invention. Other receptors, or other cell-surface characteristic determinants on antigen presenting cells may also be used as targets for the targeted antigens of the present invention. The receptor or other characteristic determinant need not be directly involved in the immune response.

C5a receptor. This receptor is preferred for use according to the present invention. It is present on PMNs, macrophages, dendritic cells, smooth muscle cells and some mast cells. A number of biological activities have been ascribed to C5a, mostly associated with inflammatory and immune responses. According to a preferred embodiment, this invention relies on the capability of C5a, as a targeting ligand, to specifically bind to its cognate receptor, so as to activate antigen presenting cells, including macrophages, monocytes and dendritic cells, through a G protein-mediated signal transduction pathway. Subsequent to signal transduction, the receptor/ligand complex is internalized. In the case of dendritic cells, C5a has been shown to induce a Th1 type response.

IFNγ receptor. The interferon γ receptor is expressed on macrophages, monocytes, dendritic cells, other APCs, some B cells, fibroblasts, epithelial cells, endothelium, and colon carcinoma cells. IFNγ binding to its receptor induces macrophage and dendritic cell activation, B cell differentiation, and expression of MHC class I and class II molecules in many cell types. The receptor is involved in signal transduction pathways. IFNγ is mainly produced in the body by activated T cells, particularly during the generation of Th1 type response. It is also produced by CD8+ cytotoxic T lymphocytes following recognition of antigen associated with MHC class I and by natural killer cells stimulated with TNFα and microbial products (Barclay et al. 1993,).

CD 21 (C3d receptor). CD 21 is the receptor for the C3d complement fragment. It is a receptor for the Epstein-Barr virus and may be an important interferon α receptor (Barclay et al., supra). CD 21 is expressed on B cells, follicular dendritic cells, other APCs, pharyngeal and cervical epithelial cells, and some thymocytes. It is involved in activation and proliferation of B cells through a signal transduction mechanism and it has been associated with increases in antigen presentation activities by those cells.

CD 64 (FcγRI receptor). CD 64 is a high affinity receptor for IgG, the only known receptor that binds monomeric IgG (Barclay et al., supra). This receptor is found on macrophages, monocytes and other immune cell populations treated with IFNγ. The IgG₁ binding site resides in the CH2 domain. IFNγ strongly upregulates expression of this receptor, which is the primary receptor involved in antibody-dependent cell mediated cytotoxicity reaction, and phagocytic activity by these cells.

CD (FcεRII receptor). CD23 is a low affinity receptor for IgE (not related to the high affinity IgE receptor found on basophils and mast cells). It is found on some B cell populations, macrophages, eosinophils, platelets, and dendritic cells (Barclay et al., supra). CD 23 mediates IgE dependent cell mediated cytotoxicity and phagocytosis by macrophages and eosinophils, and binding of IgE immunocomplexes increases the efficiency of antigen processing and presentation by some ACPs, through a signal transduction mechanism that includes the p59 fyn tyrosine kinase. The ligand for CD 23 is the Cε3 domain of IgE.

As mentioned above, the APC-targeted antigens of the present invention comprise at least one antigenic moiety and at least one targeting moiety. The targeting moiety can be derived from naturally-occurring ligands for a selected receptor on an APC, or analogs and derivatives of such ligands. For instance, the C5a receptor is a preferred receptor for use in practicing the present invention. Naturally-occurring C5a can be utilized as the targeting moiety in the APC targeted activating antigens of the invention. However, native C5a is not preferred for use as the targeting moiety as it induces a myriad of pro-inflammatory responses which may be undesirable side effects. In particularly preferred embodiments of the invention, C-terminal C5a agonist analogs capable of C5a receptor binding and signal transduction in a response selective manner are utilized. Such analogs are described in detail in U.S. Pat. Nos. 5,96,230 and 5,942,599 to Sanderson et al., and commonly-owned U.S. application Ser. No. 08/299,285, the entire disclosure of which is incorporated by reference herein.

An exemplary C5a C-terminal decapeptide agonist preferred for use in the present invention is:

| | |
|---|---|
| YSFKPMPLaR | (SEQ ID NO:1) |

This decapeptide is a potent agonist of naturally occurring C5a, and is preferred to naturally occurring C5a because its small size contributes to ease of synthesis and solubility. Moreover, these conformationally biased peptides are stable toward serum carboxypeptidase digestion, express level biological selectivity, and have been shown to be non-toxic in high concentrations in athymic mice.

Peptide analogs of naturally-occurring interferon γ are also contemplated for use in the present invention. Peptides corresponding to the amino terminal 39 amino acids of IFNγ have been shown to compete for binding with native IFNγ. Antibodies against this domain block biological activity, and removal of the first 10 amino terminal residues eliminates biological activity. This suggests that binding of IFNγ to its cognate receptor is mediated by this portion of the molecule. Accordingly, peptides based on this domain are contemplated to be of use for delivering antigens to APCs expressing IFNγ receptors. In this regard, it should be noted that human and mouse IFNγ are absolutely species specific in binding and activity. Consequently, for stimulating APC-mediated immune responses in mice, the mouse peptides will be utilized, and the human peptide will likewise be utilized for stimulating APC-mediated immune responses in humans. The mouse IFNγ 39 amino acid peptide analog is composed of the following sequence:

HGTVIESLESLNNYFNFFGIDVEEKSLFLDIWRNWQKDG
(SEQ ID NO: 3)

The human IFNγ 39 amino acid peptide analog is composed of the following sequence:

QDPYVKEAENLKKYFNAGHSDVADNGTLFLGILKNWKEE
(SEQ ID NO:4)

Another ligand contemplated for use in the present invention is the C3dG component of complement. This component is a 348 residue fragment derived by proteolytic cleavage from the C3b precursor (residue 955-1303 of C3; Swissprot accession p01024). C3dG can be converted to C3d (residues 1002-1303) and C3g (residues 955-1001). C3dG and C3d remain associated with non-activator surfaces and serve as opsonins for phagocytosis by macrophages and other antigen presenting cells. Cd 21 is the C3dG and C3d receptor.

The above-listed ligands exemplify the type of ligand preferred for practice of the present invention. However, it will be appreciated by those skilled in the art that other ligands may be utilized as the targeting moiety of the APC-targeted antigens of the invention. These include ligands that are already known in the art, as well as ligands that may be discovered and developed henceforth. Antibodies or antibody fragments also may be used to target APC-specific cell surface antigens.

The type of antigen that can be chosen as the antigenic moiety in the present invention can be any peptide, polypeptide or derivative thereof for which an immune response on antibody production is desired. These include but are not limited to, peptides, polypeptides (i.e. proteins) and derivatives thereof, such as glycopeptides, phosphopeptides and the like. Synthetic peptide and polypeptide derivatives or analogs, or any other similar compound that can be conjugated to a receptor-targeting moiety can be used in the present invention. Moreover, these peptides, proteins and derivatives may comprise single epitopes or multiple epitopes for generating different types of immune responses. Indeed, if an entire protein is conjugated to a targeting moiety, this protein is likely to comprise numerous epitopes, which may vary depending upon the solvent conditions and their effect on secondary and tertiary structure of the protein. Carbohydrates, nucleic acids and other non-protein substances also may be used as the antigenic moiety. Methods are available in the art for conjugating these substances to the peptide or protein targeting moiety.

Other substances that can be used as the antigenic moiety include small molecules, such as (1) metabolic byproducts (especially those that are toxic); (2) various environmental toxins or irritants (e.g., aromatic hydrocarbons, asbestos, mercury compounds and the like); (3) drugs (e.g., cocaine, heroin, nicotine, etc.) for treating addiction; and (4) venoms from snakes, spiders, or other organisms. Many of these kinds of small molecules are non-antigenic or weakly antigenic, so would be appropriate candidates for use in the present invention. Example 6 below illustrates the use of nicotine as the antigenic moiety.

In preferred embodiments of the invention, the antigenic moiety comprises agents that are weakly antigenic or non-antigenic under currently available immunization conditions. For example, nicotine falls into this category because it is a small organic compound that is non-antigenic under normal conditions. Equally, many tumor-associated antigens fall into this category, because the antigens are also expressed by normal cells. Therefore, immunological tolerance to such molecules makes it difficult to stimulate responses against such antigens. Other proteins that fall into this category include naturally occurring proteins form one species (e.g., human) for which it would be desirable to produce antibodies in another species but which are recalcitrant to antibody generation in the other species.

One well-characterized tumor antigen is a cell surface-associated mucin that is highly overexpressed and differentially glycosylated by different adenocarcinomas, including breast, pancreas, lung and prostate carcinomas. Aberrant glycosylation of MUC1 by adenocarcinomas results in the addition of some blood group carbohydrate antigens to this core protein and the exposure of epitopes which have been detected by monoclonal antibodies on the core protein that are not exposed on forms of this protein produced by normal epithelial cells. A full-length cDNA sequence of human mucin-1 (MUC1) revealed an encoded protein with an average length of approximately 1200 amino acids (depending on the length of the tandem repeat allele) with several obvious domains: an amino terminal signal peptide; a large domain made up of multiple identical 20 amino acid tandem repeats flanked by several repeats that contain degenerate sequences; a hydrophobic-spanning domain of 31 amino acids; and a cytoplasmic domain of 69 amino acids at the carboxyl terminus. The most well-characterized tumor associated epitopes described to date for MUC1 are found in the tandem repeat domain. These include carbohydrate structures and protein structures. MUC1 tumor associated epitopes are well characterized, and thus have been proposed to be used for the production of tumor vaccines using conventional methods. Exemplary compositions of the present invention comprise MUC1 epitopes, such as those set forth below, as the antigenic moiety of the APC-1 targeted antigens of the invention, to demonstrate the potential of the present invention as potent tumor vaccines.

MUC1 epitope predicted to bind class I molecules of the H-2K^{b} type has sequence homology to the juxtamembrane region of MUC1;

| | |
|---|---|
| YKQGGFLGL | (SEQ ID NO:6) |

MUC1 tandem report has the sequence:

| | |
|---|---|
| GVTSAPDTRRAPGSTAPPAH | (SEQ ID NO:7) |

The components comprising the APC-targeted antigens of the invention can be made separately, then conjugated. For example, a small peptide analog, such as the above-described C5a agonists, may be produced by peptide synthetic methods, and conjugated to a protein which has been purified from naturally occurring biological sources. Alternatively proteins engineered for expression via recombinant methods may be used. Additionally, targeted antigens comprising peptide components (i.e., a peptide antigenic epitope conjugated to a peptide receptor ligand) can by synthesized in tandem by peptide synthetic chemistry according to known methods and as described in greater detail below. Finally, targeted antigens of the invention comprising two larger polypeptide moieties (i.e., a large polypeptide antigen linked to a large ligand) can be made by recombinant techniques. For example, DNA molecules encoding both components can be ligated together by recombinant means, then expressed as the conjugated fusion protein. Methods of making these different types of compositions are set forth in greater detail below.

B. Peptides

Oligopeptides required for the present invention may be prepared by various synthetic methods of peptide synthesis via condensation of one or more amino acid residues, in accordance with conventional peptide synthesis methods. Preferably, peptides are synthesized according to standard solid-phase methodologies, such as may be performed on an Applied Biosystems Model 430A peptide synthesizer (Applied Biosystems, Foster City, Calif.), according to manufacturer's instructions. Other methods of synthesizing peptides or peptidomimetics, either by solid phase methodologies or in liquid phase, are well known to those skilled in the art. When solid-phase synthesis is utilized, the C-terminal amino acid is linked to an insoluble resin support that can produce a detachable bond by reacting with a carboxyl group in a C-terminal amino acid. One preferred insoluble resin support is p-hydroxymethylphenoxymethyl polystyrene (HMP) resin. Other useful resins include, but are not limited to: phenylacetamidomethyl (PAM) resins for synthesis of some N-methyl-containing peptides (this resin is used with the Boc method of solid phase synthesis; and MBHA (p-methylbenzhdrylamine) resins for producing peptides having C-terminal amide groups.

During the course of peptide synthesis, branched chain amino and carboxyl groups may be protected/deprotected as needed, using commonly-known protecting groups. In a preferred embodiment, Nα-amino groups are protected with the base-labile 9-fluorenylmethyloxycarbonyl (Fmoc) group or t-butyloxycarbonyl (Boc groups). Side-chain functional groups consistent with Fmoc synthesis may be protected with the indicated protecting groups as follows: arginine (2,2,5,7,8-pentamethylchroman-6-sulfonyl); asparagine (O-t-butyl ester); cysteine glutamine and histidine (trityl); lysine (t-butyloxycarbonyl); serine and tyrosine (t-butyl). Modification utilizing alternative protecting groups for peptides and peptide derivatives will be apparent to those of skill in the art.

C. Proteins

Full-length proteins for use in the present invention may be prepared in a variety of ways, according to known methods. Proteins may be purified from appropriate sources, e.g., human or animal cultured cells or tissues, by various methods such as gel filtration, ion exchange chromatography, reverse-phase HPLC and immunoaffinity purification, among others. However, due to the often limited amount of a protein present in a sample at any given time, conventional purification techniques are not preferred in the present invention.

The availability of nucleic acids molecules encoding a protein enables production of the protein using in vitro expression methods known in the art. For example, a cDNA or gene may be cloned into an appropriate in vitro transcription vector, such a pSP64 or pSP65 for in vitro transcription, followed by cell-free translation in a suitable cell-free translation system, such as wheat germ or rabbit reticulocytes. In vitro transcription and translation systems are commercially available, e.g., from Promega Biotech, Madison, Wis. or BRL, Rockville, Md.

Alternatively, according to a preferred embodiment, a selected peptide or protein may be produced by expression in a suitable procaryotic or eucaryotic system. For example, a DNA molecule, encoding a peptide or protein component of the invention, or an entire composite targeted antigen of the invention, may be inserted into a plasmid vector adapted for expression in a bacterial cell, such as E. coli, or into a baculovirus vector for expression in an insect cell. Such vectors comprise the regulatory elements necessary for expression of the DNA in the host cell, positioned in such a manner as to permit expression of the DNA in the host cell. Such regulatory elements required for expression include promoter sequences, transcription initiation sequences and, optionally, enhancer sequences.

A peptide or protein produced by gene expression in a recombinant procaryotic or eucaryotic system may be purified according to methods known in the art. In a preferred embodiment, a commercially available expression/secretion system can be used, whereby the recombinant protein is expressed and thereafter secreted from the host cell, so as to be readily purified from the surrounding medium. If expression/secretion vectors are not used, an alternative approach involves purifying the recombinant protein by affinity separation, such as by immunological interaction with antibodies that bind specifically to the recombinant protein. Such methods are commonly used for isolating peptides and proteins.

D. Linking Separately-Made Proteins and/or Peptides

In an alternative embodiment, protein and/or peptide components of the invention are synthesized separately, then conjugated using standard methods known by those skilled in the art. For example, a synthetic peptide may be chemically coupled to a protein using m-maleimidobenzoyl-N-hydroxysuccinimide ester (MBF). This reagent cross-links amino- and carboxy-terminal thiol groups in the peptide with lysine side chains present in the protein. Alternatively, a synthetic peptide may be coupled to a protein using glutaraldehyde, a common cross-linking agent. Another methods for chemically coupling a peptide to a protein is through the use of carbodiimide and 1-(3-dimethylaminopropyl)-3-ethylcarbodiimide methiodide (EDC). As described in greater detail in Example 2, this method was used to conjugate a C5a C-terminal decapeptide analog to serum anyloid A (SAA). Methods for joining two proteins together are also available.

The peptides or proteins of the invention, prepared by the aforementioned methods, may be analyzed according to standard procedures. For example, they may be subjected to amino acid sequence analysis, mass spectra analysis or amino acid compositional analysis according to known methods.

E. General Formulae and Exemplary Compositions of the Invention

The APC-targeted antigens of the invention can comprise one or more antigenic moieties, and likewise can comprise one or more targeting moieties. Moreover, these moieties can be functionally linked in several ways. For instance, if “T” represents a targeting moiety, and “Ag” represents an antigenic moiety, the APC-targeted antigens of the present invention may be organized as follows:
Ag-T;
T-Ag;
T₁-Ag-T₂;
T₁-[Ag]ₙ-T₂ (wherein [Ag]ₙ represents a multiplicity of antigens;
Examples of the general formulas set forth above include:
Ag-C5a agonist peptide;
IFNγ peptide-Ag;
IFNγ peptide-[Ag]ₙ-C5a agonist peptide.

Other representative compositions of the invention include:
MUC1 Class I binding epitope —C5a agonist C-terminal peptide
Murine or human IFNγ peptide—MUC1 Class I binding epitope
Murine or human IFNγ peptide—MUC1 tandem repeat
MUC1 Class I epitope—-C3dG peptide
SAA-K-Ahx—C5a C-terminal peptide (Ahx=εamino hexanoic acid).

It will be appreciated by persons skilled in the art that the APC-targeted activating antigens of the invention may be adapted for inclusion of large or complex antigens. This may be accomplished, for example, by inclusion of a “spacer” (such as the K-Ahx spacer moiety in the exemplary compound above) between the antigen and the targeting moiety. Such chemical modifications are familiar to biochemists.

Cleavable linkers. In addition to the foregoing sorts of spacers and linkers, it has also been discovered in accordance with the present invention that introduction of a cleavage-prone oligopeptide between the targeting moiety and the antigen can improve the immunogenicity of the molecule. For instance, as described in greater detail in Example 5, an Arg-Arg dipeptide or an Arg-Val-Arg-Arg (SEQ ID NO: 19) tetrapeptide between the C5a peptide analog, YSFKPMPLaR (SEQ ID NO: 1) and a cytotoxic T lymphocyte (CTL) epitope of hepatitis B virus surface antigen was found to robustly elicit a CTL response to the antigen in mice, in the absence of added adjuvant. The Arg-Arg dipeptide adds a scissile bond that is susceptible to cleavage or cleavage by trypsin-like proteases (e.g., subtilisin). The Arg-Val-Arg-Arg (SEQ ID NO: 19) tetrapeptide imbues sensitivity to cleavage by furin (another trypsin-like protease). While not intending to be limited by an particular explanation of the underlying mechanism by which the robust CTL response was elicited, it is believed that the Arg-Arg or Arg-Val-Arg-Arg (SEQ ID NO: 19) protease-sensitive linker peptides facilitate cleavage of the molecular adjuvant within the APC, thereby more expeditiously freeing the antigen for further processing and presentation of the APC cell surface.

Other scissile bond dipeptides or other oligopeptides also can be inserted between the targeting moiety and the antigen. These include residues to create a peptide bond(s) that is susceptible to other cytoplasmic proteases or proteases found within any of the intracellular antigen processing organelles. Preferred sites comprise dibasic dipeptides (i.e., various dipeptide permutations comprising Arg, Lys or His) or oligopeptide cleavage sites for the trypsin family or proteases.

Another option is the creation of a dipeptide scissile bond that is susceptible to acid-catalyzed hydrolysis when exposed to the acidic conditions typically found within intracellular endosomal/lysosomal compartments of an antigen presenting cell (APC). In addition, other epitope-targeting moiety linkages are contemplated to have a similar utility in the efficiency of processing by APCs. These include sequences that are known or suspected to facilitate or enhance 1) the transport/update of epitope constructs into the rough endoplasmic reticulum (RER), 2) the association of epitope to MHC class I and/or class II determinants, 3) transport of epitope through the cis, medial, and trans Golgi apparati, and 4) entry into transport vehicles associated with MHC class I.

Thus, a variety of “cleavable linkers” can be inserted between the targeting moiety and the antigen to facilitate processing of the antigen by the antigen-presenting cells. As used herein, the term “cleavable linker” refers to any linker between the antigenic moiety and the targeting moiety that promotes or otherwise renders the molecular adjuvant more susceptible to cleavage (by proteases, low pH or any other means that may occur within or around the antigen-presenting cell) and, thereby, processing by the antigen-presenting cell, than an equivalent molecular adjuvant lacking such a cleavable linker.

II. Uses of APC-Targeted Activating Antigens

The APC-targeted activating antigens of the present invention have broad potential for clinical applications in humans and animals. As discussed above, a significant impediment to the development of vaccines and other immunotherapeutic agents is the apparent inability of particular antigens to be readily taken up and processed by antigen presenting cells. The compositions of the invention facilitate the specific delivery of an antigen to a population of antigen presenting cells, whereupon the delivery mechanism (e.g., using as the targeting moiety a receptor ligand capable of transducing a biological signal) simultaneously activates the antigen presenting pathway. of the APC. Thus, the present invention enables development of vaccines and other immunothereapeutics that can specifically target any peptide antigen or other antigenic structure covalently attached to a ligand for a receptor present on an antigen presenting cell. It is believed that antigens linked to ligands that selectively bind to and activate a particular population of APCs can not only generate an immune response, but can influence the nature of the immune response that is generated. Thus, immune responses that favor antibody, cellular, Th1 or Th2 responses, respectively, may be selectively generated. Vaccines may also be developed with an array of such targeting moieties thereby serving to target a selected antigen or antigens to several populations of APCs and simultaneously activate these and other cells involved in various immune modulatory pathways.

The ability to generate either antibody or cell mediated immune responses against different specific antigens has broad general applicability, and it is anticipated that the APC-targeted antigens of the invention will be extremely useful for these purposes. For example, antibody responses have been shown to be capable of protecting against different viral or bacterial infection, and antibodies are known to inactivate different toxins or toxic compounds that may affect the well being of humans or animals. Different cell mediated immune responses can provide protection against viral or other intracellular pathogens, and can play a role in some anti-tumor responses. It is believed that different antigen presenting cells and the context in which these cells are stimulated to present antigen (co-stimulation mediated by different ligand-receptor interactions) are important factors determining the nature of the above responses.

The targeted antigens of the present invention should find particular utility in the development of active specific immunotherapeutic agents (i.e., cancer “vaccines”) based on cancer-associated antigens. For example, it has been hypothesized that induction of strong cell-mediated immune responses (involving Th1 cells and/or cytotoxic T lymphocytes) would provide the most effective protection against various forms of cancer. A vaccination strategy utilizing the APC-targeted antigens of the invention can be designed to induce this type of response. In this regard, it is known that stimulation with some cytokines (IL-12, IFNγ) can induce predominantly Th1 type responses over Th2 type responses for certain antigens.

As a step toward developing anti-cancer vaccines for clinical use, the compositions of the invention can be used to advantage as research tools to further explore the effect of stimulating a certain population of APCs with a tumor antigen and determining the effect on an anti-tumor immune response. To this end, it should be noted that the present application exemplifies targeted antigens comprising an epitope of a particular tumor-specific antigen, Mucin-1.

Previous tumor vaccine formulations that aim to immunize patients with compounds that are identical to compounds already produced by tumors have proven to be of limited value, probably because tumors that progress have been selected for their lack of immunogenicity in their respective host (e.g., the host is tolerant to existing tumor antigens). Thus, one important challenge of producing effective tumor vaccines is generating reagents that counteract immunological tolerance to tumor-associated antigens. One purpose of the APC-targeted antigens described above is to induce in the immunized individual a is response against their tumor that is similar to that seen in individuals undergoing allograft rejection. In other words, the goal is to induce an autoimmune reaction against the tumor that is capable of destroying the tumor. The immunological parameters that regulate tolerance to tumor antigens are not well understood; nonetheless the compositions described herein have the potential to counteract this tolerance and thus induce specific immune responses that mediate tumor rejection.

The targeted antigens of the present invention will also find broad utility in the production of antibodies for use as immunodiagnostic and immunotherapeutic agents. For immunodiagnostic purposes, antibodies are widely used in various quantitative and qualitative assays for the detection and measurement of biological molecules associated with diseases or other pathological conditions. For reasons that often are not well understood, it is sometimes difficult to generate antibodies against certain biological molecules using conventional means. The compositions of the present invention provide an alternative means for inducing an animal to produce antibodies against a weakly-antigenic or non-antigenic substances. The utility of the compositions of the invention in this regard is shown clearly in Example 2, below, in connection with serum amyloid A. The appearance and abundance of this protein in the body is strongly correlated with systemic inflammatory stress, which is a condition that is very difficult to quantitate. It is believed that quantitative assays for SAA levels would be an excellent indicator of general, systemic inflammation; therefore it would be of benefit to generate antibodies against the protein in a non-human species. This protein has proved particularly recalcitrant to the generation of antibodies using conventional measures. As described in Example 2, a targeted antigen comprising SAA conjugated to a C5a peptide ligand produced a significant antibody response in mice injected with the conjugated molecule. In a similar fashion, targeted antigens comprising any weakly-antigenic or non-antigenic component of interest could be made and used to produce specific antibodies in laboratory animals, for use as immunodiagnostic reagents.

Antibodies for use as immunotherapeutic agents can also be generated using the compositions of the invention. As one example, there has been a great deal of recent interest in developing reagents capable of down-regulating or inhibiting the complement cascade to modulate local and systemic inflammatory responses. To this end, the C3a convertase, which is active early in the cascade, could provide an ideal target for complement inhibition. C3a convertase cleaves the peptide C3 into two components, C3a and C3b, and therefore must be able to access the cleavage site on C3 in order to accomplish the result. Antibodies directed toward the C3a-C3b cleavage site are expected to be effective in blocking access of C3a convertase to the cleavage site, thereby inhibiting this early step in the complement cascade. Such antibodies may be generated using a targeted antigen of the invention comprising, as the antigenic moiety, the short peptide sequence comprising the C3a/C3b cleavage site. The sequence could then be conjugated to an appropriate targeting moiety, such as the C5a C-terminal decapeptide agonists exemplified herein. Thus, the compositions would be useful to generate an immunotherapeutic agent (e.g., an antibody that blocks the activity of C3a convertase) for treating an adverse inflammatory condition.

III. Nicotine Vaccine.

In a particularly preferred use, as stated above, the compositions of the invention may be utilized to treat the addictive properties of nicotine addiction via immunization with a nicotine vaccine. The rationale to this approach is that nicotine-specific antibodies generated in response to the vaccine bind circulating nicotine outside of the central nervous system and reduce drug access to receptor sites in the brain. This peripheral site of action, along with the high specificity and affinity of nicotine antibodies, makes vaccination an attractive therapeutic approach to smoking cessation. Also, the nicotine vaccine has the potential of inducing a “memory” immune response, wherein anti-nicotine immunity may be invoked when one in exposed to nicotine, an attractive feature for ensuring long-term compliance.

Toward that end, the composition employed as a nicotine vaccine may comprise any molecular adjuvant described herein linked to a nicotine hapten in any manner described herein. Typical embodiments of such compositions include the following:

```
(a) Nic-YSFKPMPLaR (Nic-SEQ ID NO:1);
(b) Nic-YKQGGFLGLYSFKPMPLaR (Nic-SEQ ID NO:2);
(c) Nic-YKQGGFLGLRRYSFKPMPLaR (Nic-SEQ ID NO:20);
(d) Nic
          |
        YKGGFLGLYSFKPMPLaR (Nic-SEQ ID NO:2);
(e) Nic
          |
        YKQGGFLGLRRYSFKPMPLaR (Nic-SEQ ID NO:20);
(f) Nic
          |
Nic-YKQGGFLGLYSFKPMPLaR (Nic-SEQ ID NO:2); and
(g) Nic
            |
Nic-YKQGGFLFLRRYSFKPMPLaR (Nic-SEQ ID NO: 20).
```

In one embodiment, the nicotine vaccine comprises composition (a) above and is the molecular adjuvant YSFKPMPLaR (SEQ ID NO:1) covalently modified on the N-terminal end with a nicotine hapten. In yet another embodiment, the nicotine vaccine comprises any one of compositions (b)-(g) and is composed of the B cell epitope from the juxta-membrane region of MUC1 (YKQGGFLGL)(SEQ ID NO:6) modified with nicotine at the N-terminus of the peptide and/or the α-amino moiety of the indicated Lys residue. In vaccine compositions (c), (e), and (g) the nicotine-modified B cell epitope is separated with the protease-sensitive, dibasic Arg-Arg (RR) sequence. In yet another exemplary embodiment, the nicotine vaccine compositions detailed in Example 6 may be employed.

i. Peptide Synthesis and Nicotine Attachment. Oligopeptides required for the present invention may be prepared by various synthetic methods of peptide synthesis via condensation of one or more amino acid residues, in accordance with conventional peptide synthesis methods. In one preferred embodiment, as more generally described above, the peptides may be synthesized by standard solid phase orthogonal methods in which the reactive side-chain groups of the residues are protected with acid-labile groups and the amino group of each reside is protected with the base-labile 9-fluorenylmethoxycarbonyl (Fmoc) group. In this method, synthesis begins with the fully protected C-terminal residue attached to an insoluble resin supported via its carboxyl group. The Fmoc group of this first residue is deprotected with base, thereby exposing only the amino group for a coupling reaction with the carboxyl group of the second amino acid residue to form an amide (peptide) bond between the first and second residues. The Fmoc group on the second residue may be deprotected with base exposing this residue's amino group for reaction with the carboxyl group of the third residue. This cycle of deprotection and coupling is repeated until the full peptide sequence is completed. A final deprotection of the last Fmoc group with base exposes the peptide's N-terminal amino group and all other reactive side-chain groups in the peptide remain protected. This free, N-terminal amino group may then serve as at least one site to which a nicotine molecule can be attached via an amide linkage when the nicotine molecule is modified to provide the complementary carboxyl group as detailed below.

In yet another embodiment, synthesis of the peptide is performed such that it provides carboxyl groups for nicotine attachment via an amide linkage when the nicotine molecule is modified to provide the complementary amino group as detailed below. Any one of the free amino groups generated by the above route on the protected peptide can be converted into a carboxyl group by reaction with, for example, succinic anhydride or any other appropriate reagent. Other free carboxyl groups on the peptide could be provided by the side-chains of Asp or Glu residues. These residues can come from either the naturally occurring residues in the sequence or they can be added specifically for this reason during the course of peptide synthesis by any generally known method.

After its synthesis, the resin-attached, fully protected, nicotine-modified peptide is then subjected to mild acidolysis to remove the side-chain protecting groups and cleave the peptide from the resin according to standard methods. All peptides may then be purified by preparative and analytical reverse-phase HPLC according to generally known methods and characterized by amino acid compositional analysis and mass spectrometry.

ii. Modification of Nicotine. Given the ease with which free amino and carboxyl groups can be generated at various sites on the peptide as detailed above, one method of attaching nicotine haptens to the peptide is via the formation of an amide bond provided that the nicotine molecule is modified so that it presents the complementary carboxyl or amino groups. An amide bond linkage between nicotine and the peptide is attractive because it maintains the same type of covalent linkages that already exist in the peptide, thus minimizing the possibility of introducing new immunogenic character to the antigen of interest or directing immunogenic recognition away from the antigen of interest.

Accordingly, in one embodiment, nicotine may be modified to express the complementary carboxyl or amino groups necessary for amide linkage to the peptide by the synthetic routes described below. These routes will generate the carboxyl and amino groups on different sites of the nicotine molecule, which allows the opportunity of conjugating nicotine to various sites on the peptide such that different potential antigenic regions of the nicotine molecule can be exposed and presented.

The nicotine haptens, NH1 and NH2, described in sections a. and b. below each express carboxylic acid functional groups to allow amide bond formation with either the N-terminal or Lys ε-amino groups of the peptide.

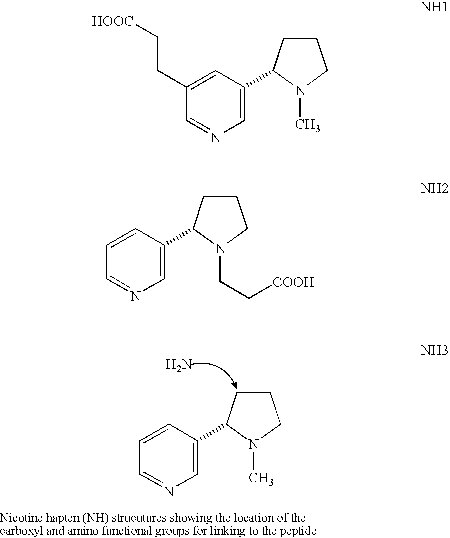

a. NH1: Carboxyl Modification of the pyridine Ring of Nicotine. As shown in Scheme 1 below, in one embodiment, the synthesis of NH1 begins with treatment of commercially available ethyl-5-bromo-nicotine (1) with 1-vinylpyrrolidinone anion followed by acid-catalyzed decarboxylation and ring closure to give 5-bromomyosmine (2). Reduction of 2 with NaBH₄/CBZ-D-proline followed by N-methylation and resolution with dibenzoyl-L-tartaric acid will provide (S)-5-bromonicotine (3). Coupling of 3 with ethyl acrylate followed by sequential hydrogenation and ester hydrolysis will yield NH1.

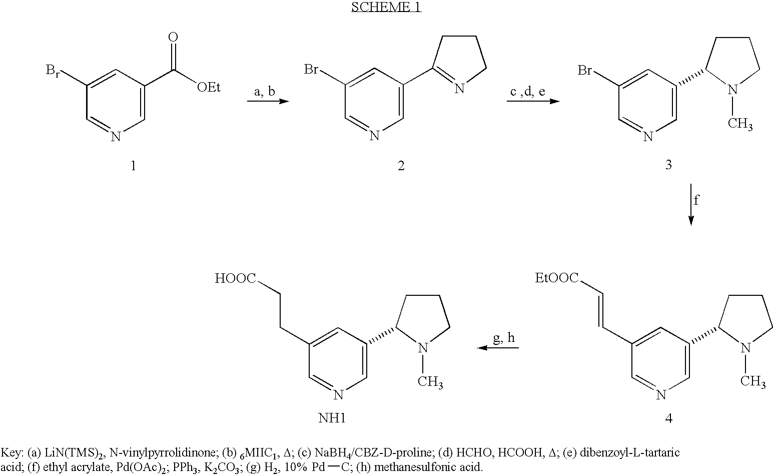

In yet another embodiment, NH1 may be prepared by first converting 3 to its iodo derivative, (S)-5-iodonicotine, via hexa-n-butylditin and a catalytic amount of tetrakis(triphenyl-phosphine)palladium followed by iodostannylation with iodine monochloride. In a further embodiment, NH1 can synthesized by producing (S)-5-ethylnicotine from 3. This intermediate is then employed in a palladium catalyzed coupling with ethyl chloroformate (52) to form the alkyne ester. As described above, sequential hydrogenation and ester hydrolysis of the alkyne ester yields NH1.

b. NH2: Carboxyl Modification of the Pyrrolidine Nitrogen of Nicotine. As shown in Scheme 2 below, the synthetic intermediate 2 described above will serve as the starting material for the synthesis of NH2. Reduction of 2 with NaBH₄, resolution with (+)-MTPA, and reductive debromination with hydrogen and palladium on carbon affords (S)-nor-nicotine (5). In yet another embodiment, 5 can be obtained in an enantio-selective synthesis starting with a chiral 2-hydroxy-3-pinanone ketimine. Treatment of 5 with β-propiolactone in hot acetonitrile will afford NH2.

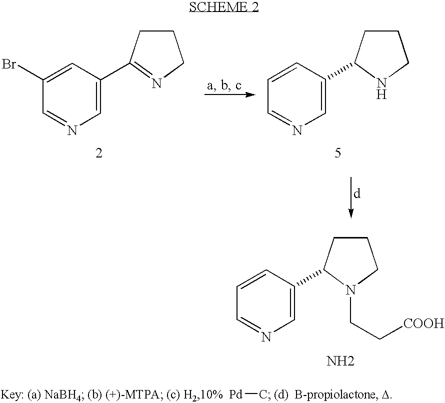

In still a further embodiment, the conversion of 5 to NH2 is performed by conjugate addition of 5 to ethyl acrylate followed by ester hydrolysis. Reductive alkylation of 5 with NaBH, and hydroxy acetic acid is another potential method to generate NH2.

c. NH3: Amino Modification of the Pyroline Ring of Nicotine. As shown in Scheme 3 below, synthesis of NH3 may begin by treatment of the commercially available pyridine-3-carboxaldehyde with methylamine to form the imine. Treatment of the imine with succinic anhydride in boiling xylene affords trans-1-methyl-4-carboxyl-5-(3-pyridyl)-2-pyrrolidone (7). Esterification and subsequent lithium aluminum hydride (LAH) reduction affords trans-3′-hydroxymethylnicotine (8). The final three steps in the synthesis of NH3; i.e., tosylation of alcohol 8, conversion to the azide, and azide reduction by LAH may be performed according to any generally known method. In yet another embodiment, NH3 may be formed by conversion of 7 to its 4-carboxamide followed by an LAH reduction of both lactam and carboxamide functional groups.

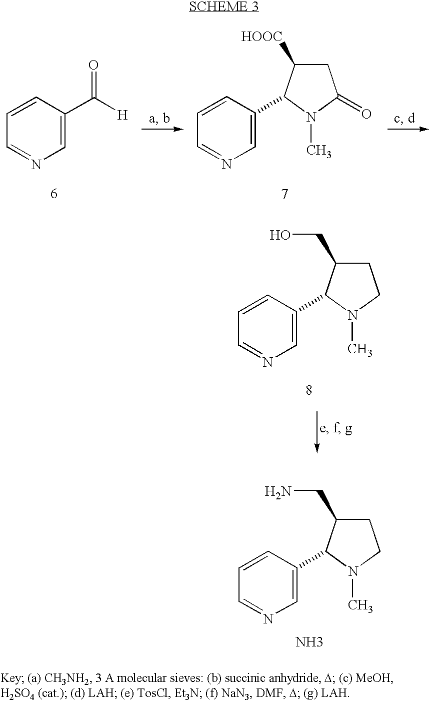

d. NH Characterization. The nicotine haptens NH1, NH2, and NH3, all reaction intermediates, and starting materials may be purified by distillation, crystallization, or flash column chromatography. These molecules may then be purified by analytical and semi-preparative HPLC and structure/composition confirmed by ¹H and ¹³C NMR, infrared spectroscopy, optical rotation, melting point, elemental analysis, and mass spectrometry or any other generally known method.

It will be appreciated by the skilled artisan that any number of variations for preparing the peptide or nicotine hapten other than the methods set forth above may be utilized. In addition, it will also be appreciated by the is skilled artisan that methods other than those detailed above for attaching the nicotine hapten to the peptide may be employed.

The following examples are provided to describe the invention in further detail. These examples are intended to illustrate the invention in greater detail. They are not intended to limit the invention in any way.

### EXAMPLE 1

### Evaluation of Mucin Epitope (MUC1/C5a Agonist) Conjugate for Recruitment and Activation of Antigen Presenting Cells (APCs) and Stimulation of an Immune Response in Mice

The C5a receptor is present on numerous antigen presenting cells, including monocytes, macrophages, dendritic cells, and other cell types. In this example, a composite peptide comprising a mucin epitope (MUC1) functionally linked to a decapeptide agonist analog of C5a corresponding to the C-terminal effector region of the natural favor was evaluated for its ability to activate the APCs thereby stimulating an immune response in mice. This evaluation is based on the known property of C5a receptors to internalize and recycle in the antigen presenting cell, thereby acting as ideal candidates for delivering antigens to and simultaneously activating signals in the APCs. Because C5a receptors are particularly common on macrophages, monocytes and dendritic cells, it is believed that the use of a C5a agonist analog to bind C5a receptors will result in preferential activation of these APCs.

i. Abbreviations. Except where noted, the single letter designation for the amino acid residues is used: alanine is A; arginine is R; asparagine is N; aspartic acid is D; cystine is C; glutamine is Q; glutamic acid is E; glycine is G; histidine is H; isoleucine is I; leucine is L; lysine is K; methionine is M; phenylalanine is F; proline is P; serine is S; threonine is T; tryptophan is W; tyrosine is Y; and valine is V. Upper case letters represent the L-amino acid isomer and lower case the D-isomer.

ii. Peptide synthesis, Purification and Characterization. The following peptides were synthesized according to standard solid-phase methodologies on an Applied Biosystems (Foster City, Calif.) model 430 A peptide synthesizer and characterized as previously described (7):
(1) The antigenic “juxta-membrane” (JM) epitope of the human mucin-1 (MUC1), YKQGGFLGL (SEQ ID NO:6);
(2) The C5a C-terminal decapeptide agonist analog, YSFKPMPLaR (SEQ ID NO:1);
(3) The composite peptide YKQGGFLGLYSFKPMPLaR (SEQ ID NO:2), in which the JM epitope is positioned toward the amino terminus and the C5a peptide is positioned toward the carboxyl terminus; and
(4) The composite peptide YSFKPMPLaRKQGGFLGL (SEQ ID NO:5), in which the JM epitope of MUC1 is positioned toward the carboxyl terminus and the C5a analog is positioned toward the amino terminus.

Peptide 3 retains C5a biological activity, whereas peptide 4 does not because the biologically important carboxyl terminal end of the C5a analog is blocked by the presence of the mucin epitope. As such, peptide 4 serves as a control to determine the importance of the C5a biological activity to the effectiveness of these peptides for immunization purposes.

Syntheses were performed on a 0.25 mmol scale on O-hydroxymethylphenoxymethyl polystyrene (HMP) resins (0.88 meq/g substitution). N^{α}-amino groups were protected with the base-labile-9-fluorenylmethyloxycarbonyl (Fmoc) group. Side-chain functional groups were protected as follows: Arg (Pmc or 2,2,5,7,8-pentamethylchroman-6-sulfonyl); Asp (Ot-butyl ester); Cys, Gln & His (Trt or trityl); Lys (Boc or t-butyloxycarbonyl); Ser & Tyr (t-butyl). Synthesis was initiated by the in situ coupling of the C-terminal residue (N^{α}-Fmoc-L-Arg(Pmc)) to the HMP resin in the presence of excess N-N′-dicyclohexylcarbodiimide (DCC) and 1-hydroxybenzotriazole (HOBT) with 4-dimethylaminopyridine (DMAP) as a coupling catalyst. Peptide chain elongation was accomplished by repetitive Fmoc deprotection in 50% piperidine in NMP followed by residue coupling in the presence of 2-(1H-benzotriazol-1-yl)-1,1,3,3-tetramethyluronium hexafluorophosphate (HBTU).

Side-chain deprotection and cleavage from the resin were achieved in a single step acetolysis reaction by stirring the peptide-resin in a solution of 84% trifluoroacetic acid (TFA), 6% phenol, 2% ethandithiol, 4% thioanisole, and 4% water for 1.5 hr at room temperature. Free peptide was precipitated from this solution by adding cold diethyl ether. The mixture was filtered through a scintered glass Buchner funnel (medium porosity) and the peptide/resin washed twice with cold ether to remove the thiol scavenger. The peptide was extracted by swirling the peptide/resin in the funnel with 20-30 ml aliquots of 10% acetic acid followed by filtration. The extraction aliquots were combined, frozen and lyophilized to yield the powdered form of the crude peptide.

Peptides were purified by preparative and analytical reverse-phase HPLC on columns packed with C₁₈-bonded silica. The details of this procedure have been described by (4). All peptides were characterized by amino acid compositional analysis and fast atom bombardment mass spectrometry (FAB-MS).

iii. Animal Models. The strains of mice used for this example were inbred females 6 to 12 week old C57Bl6(H-2^{b}) and Balb/c (H-2^{d}), which were obtained from Jackson labs (Bar Harbor, Me.). These two strains which differ in H-2 haplotypes, were used in this example to demonstrate that the observed antibody response were not a result of the selection or creation of an unique immunogenic epitope characteristic of the sequence of the proteins of the MHC class I and class II molecules important for antigen processing in one mouse strain or another. The MUC1 peptide selected for these studies contained a motif that may bind to the H-2K^{b} molecule of the C57Bl6 mice; therefore, a strain of mouse that lacked this class I molecule binding motif (Balb/c) was also studied to determine the relative contribution of the class I binding motif to the antigen presentation properties of these peptides.

iv. Immunization protocol. Preimmune sera were obtained from mice, which were subsequently immunized intraperitoneally with 100 μg of the indicated peptide with RIBI adjuvant (MPL+TDM+CWS) (Sigma Immunochemicals). Animals were boosted twice at two week intervals using the same injection procedure. Sera were obtained following three immunizations (at 6 weeks).

v. Analysis of serum antibody responses. For radioimmunoassay (RIA), anti-peptide antibodies were determined, before and at different time points after immunization, in 96-well microtiter plates (Dynatech Laboratories, Inc.). Plates were coated with 50 μl of a 100 μg/ml appropriate peptide in phosphate-buffered saline (PBS) pH 7.4 solution overnight at 4° C. The wells were blocked by incubation with 5% dry milk in PBS pH 7.4 for at least two hours. Anti-peptide antibody titers were determined using serial dilutions of sera. The sera were diluted with PBS containing 0.05% Tween-20, pH 7.4 (washing buffer) and 50 μl of each dilution was incubated at 37° C. for 1 hour. The wells were then drained, washed 4 times with PBS-Tween and 50 μl of ¹²⁵I-goat anti-mouse Ab (1-2×10⁴ cpm/well) was added and incubated for 1 hr at 25° C. After washing, specific radioactivity was recorded in a gamma counter (1272 CliniGamma, LKB).

Anti-peptide antibody isotype titers were determined by enzyme-linked immunosorbent assay (ELISA) carried out in 96-well microtiter plates. The plates were coated with 100 μg/ml of appropriate peptide in PBS, pH 7.4, and incubated overnight. The wells were blocked with 5% dry milk in PBS pH 7.4 for at least two hours. Anti-peptide titers were determined using serial dilutions of sera as described above. After the plates were washed 4 times, 50 μl of a 1:100 dilution of rabbit anti-mouse IgA, IgG1, IgG2a, IgG2b, IgG3 and IgM (Zymed) was added to each well and incubated at 25° C. for 1 hour. The plates were washed 4 times with washing buffer and 50 μl of 1:500 goat anti-rabbit conjugated to peroxidase (Zymed) was incubated at 37° C. for 1 hour. Again, the plates were washed 4 times with washing buffer and bound enzyme was detected by the addition of 50 μl 1 mg/ml p-nitrophenyl phosphate (Sigma) in 10% diethanolamine (Sigma) pH 9.4. The reaction was stopped by the addition of 50 μl of 0.5 M sodium hydroxide and absorbance values (A₄₀₅) were determined on Titertek Multiskan (Flow Laboratories, Irvine, Scotland).

vi. Experimental groups. Experimental groups were as follows:
Group A. mice immunized with peptide (1)
Group B. mice immunized with peptide (2)
Group C. mice immunized with peptide (1) plus peptide (2)
Group D. mice immunized with peptide (3)
Group E. mice immunized with peptide (4).

The results of the experimental protocols are set forth in FIGS. 1 and 2. As can be seen in the Figures, the mice in Groups A, B, C and E produced no appreciable increase in antibody response to inoculation with MUC1 epitope (Group A), C5a agonist peptide (Group B) MUC1 epitope combined with, but not conjugated to, C5a agonist peptide (Group C), or MUC1 epitope conjugated to the C5a agonist peptide at its C-terminus, rather than its N-terminus (thereby blocking C5a biological activity) (Group E). Only mice inoculated with the MUC1 epitope/C5a agonist peptide conjugate of the present invention (Group D) generated an appreciable antibody response. Furthermore, this stimulation was significant. It is clear from these results that inoculation with the conjugated MUC1 epitope/C5a agonist peptide was far more efficient in stimulating a general immune response (i.e., production of antibodies) than was inoculation with either peptide alone, or both peptides together, but not conjugated, or peptides conjugated in the opposite orientation.

There are several significant conclusions that can be drawn based on these results. The fact that both Balb/c and C57Bl6 mice showed antibody responses to peptide 3 suggests that the antigen presenting effect is not restricted by MHC haplotype. The fact that immune responses were not produced to peptide 4, or to mixtures of peptide 1 and 2, but that substantial responses were produced to peptide 3, suggest that the effect is mediated by the C5a moiety of the peptide and that the immune response results from the simultaneous delivery of antigen peptide and C5a mediated activation signals to antigen presenting cells.

The isotypes of the anti-peptide antibodies produced in the immunized mice were determined (FIG. 3) and were found to consist of IgM, IgG2a, and IgG2b. This suggests that the immunogenic peptide is producing T cell-dependent responses, which generally require antigen processing and presentation. Data presented in FIG. 4 show that the antibody response to peptide 3 includes a high percentage of antibodies that are specific for the MUC1 epitope that was antigen moiety of these studies.

### EXAMPLE 2

### Evaluation of Serum Amyloid A/C5a Peptide Conjugates for Recruitment and Activation of APCs and Stimulation of Immune Response in Rats

Serum amyloid A is an acute-phase stress response protein generated by the liver. Along with other acute phase proteins, SAA is secreted in response to systemic inflammatory stress as a protective measure. SAA is of interest because it appears to be an excellent indicator of general, systemic inflammation, which is a phenomenon that is very difficult to quantitate. Because serum levels of SAA have been observed to parallel the rise and fall of the systemic inflammatory response, quantization of serum levels of this peptide would provide an effective means of assessing inflammation. One way to accomplish this is to develop antibodies against SAA that could be used for quantitation such as in an ELISA assay. However, SAA has been particularly recalcitrant to the generation of antibodies against it. In this example, an evaluation was made of the ability of SAA conjugated to a C5a C-terminal analog (as described in Example 1) to activate antigen producing cells and produce an antibody response in rats.

i. Production and preparation of proteins and peptides. The C-terminal C5a analog K-Ahx-YSFKPMPLaR (SEQ ID NO:8) (AhX is aminohexanoic acid, which is a linear aliphatic spacer moiety) was produced as described in Example 1. The aliphatic spacer moiety was included to separate the critical receptor-binding C5a analog from the bulky protein to be attached to the amino terminus.

Serum amyloid A was conjugated to the C5a peptide analogs according to the following method. SAA (100 μg) was reacted with a 50-fold molar excess of a water soluble carbodiimide, 1-(3-dimethylaminopropyl)-3-ethylcarbodiimide methiodiide (EDC), in 200 μl of phosphate buffered saline, pH 7.5, at room temperature for 30 minutes. A 50-fold molar excess of the peptide (K-Ahx-YSFKPMPLaR) (SEQ ID NO:8) and a 100-fold molar excess of a base diisopropylethyl amine (DIEA) were added to this solution. Water was added to the solution to bring the reaction mixture to a volume of 400 μl. This solution was stirred overnight at room temperature and then lyophilized to a dry powder. The powder was diluted to the appropriate volume with water to generate the stock mixture used for inoculating the animals.

ii. Experimental protocols. Rats were injected intraperitonealy with an inoculant comprising the SAA/C5a peptide conjugates in phosphate-buffered saline with or without RIBI adjuvant. Booster injections were given two and five weeks after the initial injections. The rats were sacrificed seven weeks after the initial injection and anti-mucin antibody production was assessed from the serum titers, as described in Example 1.

Significant anti-SAA antibody was produced from both groups of rats, whether or not RIBI adjuvant was included in the inoculation. As visualized by gel electrophoresis and autoradiography of anti-SAA antibody eluted from the plate assays, it appeared that anti-SAA antibody titers were essentially equivalent, or slightly higher, in rats inoculated with SAA/C5a peptide conjugate in the absence of RIBI adjuvant as compared to the same inoculation without the adjuvant. Thus, antigenic conjugates comprising the C5a peptide analog are useful for generating antibodies against large proteins, as well as against smaller peptide fragments, such as those described in Example 1. Moreover, the successful generation of anti-SAA antibodies utilizing this method is particularly promising for purposes or producing antibodies against weakly- or non-antigenic peptides or proteins.

### EXAMPLE 3

### Production and Characterization of Site-Directed Neutralizing Antibodies Specific for a Peptide (κR(33-52) from the Predicted Amino-Terminal Region of the Human Kappa Receptor

Receptors for human opioid peptide hormones have been described on numerous cell types. The receptors for μ, κ and δ ligands have recently been cloned from genomic and cDNA libraries derived from normal tissue and cell lines. Considerable homology exists among the μ, κ and δ receptors, except for the N-terminal regions of the receptors. The N terminal region of the human kappa receptor (amino acid residues 1-100) is relatively hydrophilic and would be predicted to be exposed on the surface of the cell membrane. A 20 residue peptide [κR(33-52)], was chosen and used to raise a site directed peptide specific polyclonal antibody (5).

The method of production of a polyclonal antiserum in rabbits using the molecular adjuvant, C5a-agonist peptide conjugated to the κR epitope is set forth below. The binding specificity and biological activities of the resulting polyclonal antiserum raised to the predicted extracellular region of the human kappa receptor (κR) are also described below.

i. Construction of Targeted-Immunogen. A peptide construct consisting of the κR(33-52) (FPGWAEPDSNGSAGSEDAQL) (SEQ ID NO:9) covalently attached to the N-terminal end of a conformationally biased, C5a complement fragment agonist analogue peptide (YSFKPMPLaR) (SEQ ID NO:1) was synthesized according to the methods in Example 1 and as previously reported (7).

ii. Preparation of anti-κR(33-52) Antiserum and Peptide-Specific ELISA. Rabbits were immunized s.c. with 500 μg of FPGWAEPDSNGSAGSEDAQLYSFKPMLaR (SEQ ID NO:10) construct in complete Freund's adjuvant (GIBCO, Grand Island, N.Y.) on day 0 followed by booster injections on days 30 and 60 in incomplete Freund's adjuvant. Serum was collected starting 75 days after the initial immunization.

The presence of anti-peptide antibody was determined by using a peptide specific ELISA utilizing the free κR(33-52) peptide as previously described (8). Anti-κR(33-52) and normal rabbit γ-globulin (RGG) were purified by protein A Sepharose chromatography (Sigma) (8) prior to use.

iii. Cells and culture conditions. The neuroblastoma cell SK-N-SH (HTB 11), ductal breast cell carcinoma T47D (HTB 133), Jurkat T cell leukemia, (TIB 152), U937 histolytic lymphoma (CRL1593), THP 1 human monocyte (TIB 202), EBV-transformed B cells SKW 6.4 (TIB 215) and CESS (TIB 190) (American Type Culture Collection, Rockville, Md.) were cultured in DMEM or RPMI 1640 supplemented with 10% fetal calf serum, 25 mM HEPES, 1 mM L-glutamine, 2 mM Na pyruvate, 50 U penicillin and 50 μg/ml streptomycin. The human neuronal precursor cells NT2 (Stratagene, La Jolla, Calif.) were cultured in Opti-MEM (Gibco) supplemented as above. All cultures were incubated at 37° C. in a humidified chamber with 7.5% CO₂.

Peripheral blood derived mononuclear cells were obtained from healthy male and female volunteers, isolated by Ficoll-Hypague™ density gradient centrifugation and enriched for macrophage by adherence to plastic.

iv. Flow Cytometry. Single-color flow cytometry analysis of cells (1×10′) in PBS containing 1% bovine calf serum and 0.1% sodium azide (staining buffer) were preincubated 30 minutes at 4° C. in the presence of 20% normal human serum. The cells were washed and incubated with anti-κR(33-52) or RGG for 30 minutes at 4° C., washed and labeled with PI-conjugated donkey (Fab′)₂ fragments of antirabbit IgG (Zymed, S. San Francisco, Calif.) for 30 minutes at 4° C. (8). For dual color analysis FITC-conjugated anti-CD3 or anti-CD14 (Pharmingen, San Diego, Calif.) were also included in the second step. Cells (1×10⁴) were analyzed on a FACScan (Becton Dickinson, Mountain View, Calif.) and data were analyzed with the Cell Quest software as previously described (8).

v. Measurement of cell proliferation. Peripheral blood mononuclear cell (PBMC) were pulsed on day 2 of culture with ³H-thymidine and 18 hours later the cells harvested on glass fiber filters and processed for scintillation counting. Experiments were performed three times and each sample done in triplicate.

vi. Measurement of IgG Secretion. Relative IgG levels in culture supernatants were determined by indirect ELISA as previously described (9). Supernatant derived from PBMC cultures were collected after 10 days and assayed for the presence of IgG. Numbers represent the mean CPM+/−SD from triplicate samples. Experiments were performed at least three times.

vii. Characterization of Anti κR Peptide Antiserum. Serum from rabbits immunized with the κR(33-52) YSFPMPLaR (SEQ ID NO:10) construct and normal rabbit serum were assayed for the ability to recognize plate bound κR(33-52) (SEQ ID NO:9) in ELISA. The results show that serum from rabbits immunized with the κR(33-52) YSFPMPLaR (SEQ ID NO:10) construct bound free κR(33-52) peptide (SEQ ID NO:9) in a dose dependent fashion. The titer was approximately 105. In contrast, serum from unimmunized rabbits failed to bind this peptide. Serum samples from immunized and unimmunized rabbits were subjected to protein A-Sepharose chromatography and the column eluates were assessed for κR(33-52) (SEQ ID NO:9) specific antibody. The results indicate that protein A-purified antibody derived from rabbits immunized with the κR(33-52) YSFPMPLaR (SEQ ID NO:10) construct binding to free κR(33-52) (SEQ ID NO:9) was detectable at antibody concentrations less than 0.1 ng/ml. In contrast, RGG failed to bind the free peptide. The results from multiple bleedings indicated that the ED₅₀ titer ranged between 1-10 ng/ml. These results indicate that rabbits immunized with κR(33-52) YSFPMPLaR (SEQ ID NO:10) contained high titer, κR(33-52) peptide specific antibody.

viii. Binding of anti-R (33-52) antibody to cells expressing human κR. To determine whether the polyclonal anti-κR(33-52) antibodies bound to cells expressing the κR, a variety of mononuclear cells lines and normal human mononuclear cells were first assayed for the presence of the κ receptor specific mRNA by RT-PCR. RNA samples isolated from neuronal cell lines NT2, U937, Jurkat, T47D, normal human PBMC, and enriched human macrophage were subjected to RT-PCR analysis with 5′ sense and 3′ antisense primers specific for the 3′ region of the cloned κR and B-actin. All of the cell lines or cell fractions, except for the T47D cell line, were positive for the K-receptor specific PCR product, as expected based on the primer sequences used (5).

Experiments were performed to determine whether anti-κR(33-52) bound to cells expressing κR specific mRNA. The results of single color flow cytometric analysis for several cell lines are shown in Table 2. Flow cytometric measurements were conducted with human cell lines representative of macrophage (U937), T lymphocytes (Jurkat), and B lymphocytes (SKW 6.4 and CESS). The results indicate that anti-κR(33-52) bound all three cell types. Anti-κR(33-52) bound to U937 cells to the greatest extent (MFI=231) compared to normal RGG (MFI=38). As used herein MFI refers to mean fluorescence intensity. Comparison of anti-κR(33-52) and RGG binding to the Jurkat line indicated approximately a 3-fold shift in MFI (MFI=18 vs. MFI=6). Similar results were obtained with the two B lymphocyte-like cell lines (SKW 6.4 and CESS). Comparison of anti-κR(33-52) and RGG binding to the SKW 6.4 line indicated approximately a 3-fold shift in MFI (MFI=19 vs. MFI=6). The neuronal cell line was also specifically bound by the anti-κR(33-52) as indicated by a 3-fold shift in the MFI over the RGG. Finally, based on the lack of expression of κR-specific mRNA from the human breast carcinoma cell line (T47D), this cell line was assessed for its ability to bind to anti-κR(33-52) by flow cytometric analysis. The lack of a κR expression on T47D cells was confirmed by the fact that anti-κR(33-52) and RGG bound to these cells in an almost identical fashion. As a positive control, anti-κR(33-52) and RGG were assessed for their ability to bind to an additional human macrophage-like cell line (THP 1). Comparison of anti-κR(33-52) and RGG binding to this cell line resulted in a significant shift in MFI (MFI=190 vs. MFI=8). These results confirm the specificity of anti-κR(33-52) for the human κR.

TABLE 1
Selected cell type binding of anti-κR(33-52)
antibodies produced in rabbits immunized with C5a-agonist
peptide conjugated to the κR(33-52) sequence as assessed
by single channel color flow cytometric analysis.

| | | Mean | Channel Intensity |
|---|---|---|---|
| Cell Line | Cell Type | RGG | anti-κR Ab |
| NT2 | Neuronal | 9 | 19 |
| U937 | Macrophage | 38 | 231 |
| Jurkat | T-lymphocyte | 6 | 18 |
| SKW 6.4 | B-lymphocyte | 6 | 19 |
| CESS | ″ | <10 | <10 |
| Controls | Human breast | ˜3 | ˜3 |
| T47D (negative) | Carcinoma | | |
| THP1 (positive) | Macrophage | 8 | 190 |

Analysis of intact human PBMC indicated that these cells express mRNA for a “κ-like” R (5). Dual color flow cytometric analysis was utilized to assay for the binding of anti-κR(33-52) to normal human macrophage (CD14+) and T lymphocytes (CD3+). It was observed that both macrophage and T lymphocytes bound anti-κR(33-52) antibody. Anti-κR(33-52) and RGG were assessed for binding to CD14+ PBMC. The results indicate that anti-κR(33-52) bound CD14+ cells with a 15-fold increase compared to normal RGG (MFI=320 vs. MFI=21). Anti-κR(33-52) was also found to bind CD3+ cells (MFI=19 vs. RGG MFI=3) albeit less than CD14+ cells. These results indicate that anti-κR(33-52) binds normal PBMC-derived mononuclear cells as well as mononuclear cell lines, which express κR-specific mRNA.

ix. Neutralization of U50,488H-mediated suppression of lymphocyte proliferation by anti-κR(32-52) antibody in vitro. The results of published studies have shown that opioid peptide-induced regulation of in vitro immune responses can occur via specific receptor-ligand interactions. More specifically, it has been shown that the κR-selective agonist U50,488H is capable of suppressing SAC-induced lymphocyte proliferation by human PBMC cultures (6). The inhibition of lymphocyte activation by U50,488H has also been shown to be reversed by the κR-selective antagonist nor-BNI. To determine whether anti-κR(33-52) was capable of acting as an κR selective antagonist and neutralizing U50,488H-mediated suppression, PBMC cultures were preincubated with various concentrations of protein A purified anti-κR(32-52) prior the addition of SAC and U50,488H. U50,488H suppresses SAC-induced lymphocyte proliferation in a dose dependent fashion (5). Maximal suppression was obtained when U50,488H was used at a concentration of 10⁻⁶ M. PBMC cultures were preincubated with various concentrations of anti-κR(33-52) (1-50 μg/ml), followed by the addition of U50,488H plus SAC, and proliferation measured on day 3 of culture. Anti-κR(33-55) was found to neutralize U50,488H-mediated suppression of SAC-induced lymphocyte proliferation in a dose dependent fashion. In contrast, identical concentrations of normal RGG failed to inhibit κR selective agonist mediated immunosuppression.

Since SAC has been shown to induce both T and B lymphocyte proliferation, similar experiments were conducted with the T cell mitogen PHA. Anti-κR(33-52) was also able to neutralize the ability of U50,488H to suppress mitogen-induced T cell proliferation. U50,488H (10⁻⁶ M) suppressed PHA-induced T cell proliferation by 85%. This suppression was reversed by preincubating the cells with anti-κR(33-52). Preincubation of PBMC with normal RGG failed to block U50,488H-mediated suppression of T cell proliferation.

Anti-κR(33-52) does not appear to directly modulate lymphocyte proliferation. The co-culture of PBMC with anti-κR(33-52), in the absence of mitogen, failed to stimulate the cells above the media control. Moreover, the combination of anti-κR(33-52) and PHA or SAC did not result in increased cell proliferation compared to PBMC cultures receiving mitogen only.

x. Neutralization of U50,488N-mediated suppression of IgG synthesis by anti-κR(32-52) antibody in vitro. In addition to lymphocyte proliferation, U50,488H is a potent inhibitor of SAC-induced IgG synthesis in human PBMC cultures (6). To determine whether anti-κR(32-52) was capable of neutralizing the suppression of IgG synthesis, PBMC were preincubated with anti-κR(32-52) followed by the addition of U50,488H and SAC, and IgG levels measured on day 10. Results indicate that U50,488H at 10⁻⁸ M and 10⁻⁷ M inhibited IgG synthesis by 67% and 85% respectively (5). The inclusion of anti-κR(32-52) in culture was found to neutralize suppression of SAC induced IgG synthesis in a dose dependent manner. In contrast, similar concentrations of normal RGG failed to neutralize the observed suppression.

To assess the specificity of anti-κR(32-52) antibody, PBMC were incubated with specific antibody or RGG followed by co-culture with U50,488H or the μ receptor selective agonist (DAGO) and IgG production measured by ELISA. The results indicate that, whereas, anti-κR(32-52) neutralized U50,488H-mediated inhibition of SAC-induced IgG synthesis, anti-κR(32-52) was unable to neutralize DAGO-mediated suppression of IgG synthesis.

These results indicate that in addition to binding lymphocytes and macrophage, anti-κR(32-52) is capable of neutralizing the ability of a κR selective agonist (U50,488H), but not a μR selective agonist (DAGO). Additionally the antibody demonstrated significant inhibition of both lymphocyte proliferation and differentiation to antibody synthesis. These results further demonstrate the specificity of anti-κR(33-52) for the human kappa receptor.

As can be seen from the antibody binding data presented above, the site directed polyclonal antibodies raised in rabbits using the C5a-agonist form of the molecular adjuvant conjugated to the κ receptor sequence were capable of binding to normal human cells and cell lines expressing mRNA specific for the human κ receptor. Flow cytometric analysis of a neuronal cell line (NT2), normal blood-derived CD14+monocytes, monocyte-like cell lines (U937 and THP1), normal blood derived CD3+ T cells and a T cell line (Jurkat), and human B cell lines (SKW6.4 and CESS) revealed that the cells were all bound by anti-κR(33-52) in a specific manner. The anti-κR(33-52) did not bind to a cell line determined not to express mRNA for the human κ receptor.

Anti-κR(32-52) was found to specifically neutralize κR-selective agonist (U50,488H)-mediated inhibition of lymphocyte activation. The antiserum was found to neutralize, in a dose dependent manner, U50,488H-mediated inhibition of: 1) SAC-induced lymphocyte proliferation; 2) PHA-induced lymphocyte proliferation and; 3) SAC-induced IgG synthesis. In contrast, DAGO-mediated suppression of SAC-induced IgG production was not affected by anti-κR(32-52). These results suggest that this site directed polyclonal antiserum specifically interacts with the human κR on PBMC. The results presented indicate that polyclonal anti-κR(32-52) antibodies interact with the exposed N-terminal region of the κR. While this antiserum effectively blocked U50,488H-mediated lymphocyte activation, it did not activate macrophage or lymphocytes.

While anti-K opioid receptor antibodies are exemplified above, conjugation of C5a agonist peptide to peptides corresponding to p and A specific peptides has resulted in the successful generation of specific antibodies to the p and A epitopes.

### EXAMPLE 4

### Comparison of Immunogenicity of Epitope-C5a Agonist Constructs with Epitope-KLH Conjugates

The following experiment was performed in order to compare the potency of the molecular adjuvant of the present invention with a widely used method for enhancing the immune response to peptide epitopes. The objective was a direct comparison of the response to a construct of MUC1 epitope-C5a agonist and the same epitope conjugated to keyhole limpet hemocyanine (KLH) in mice. The results are summarized in Table 2.

TABLE 2
MUC1 Specific Ab Isotype Titers Produced with Different Immunogens.
Ab Isotypes and Titers^{a}

| | IgA | IgG1 | IgG2a | IgG2b | IgG3 | IgM |
|---|---|---|---|---|---|---|
| YKQGGFLGLYSFKPM | 0 | 0 | 1260 | 1780 (5/5) | 0 (5/5) | 6310 (5/5) |
| PLaR^{b} | | | | | | |
| (SEQ ID NO: 2) | | | | | | |
| YKQGGFLGL-KLH^{c} | 0 | 100 (2/5) | 0 | 0 | 0 | 5010 (4/5) |
| (SEQ ID NO: 6-KLH) | | | | | | |

^{a} Sera were screened against MUC1 peptide and mean titer values of responders are shown. Parentheses indicate the number of responders. Ab titer is defined as the sera dilution within the linear range at which specific reactivity is lost.

^{b} Five C57BL6 mice were immunized and boosted with YKQGGFLGLYSKFPMPLaR (SEQ ID NO:2) and sera were obtained as indicated in the Material and Methods section. Standard error of responder titer values was less than 32% for all isotypes.

^{c} Five C57BL6 were immunized and boosted with YKQGGFLGL-KLH (SEQ ID NO:6-KLH) and sera were obtained as indicated in the Materials and Methods section. Standard error of responder titer values was less then 25% for IgM and less than 40% for IgG1.

A similar experiment was performed in rabbits. The immunogens used in rabbits were the κ- and μ-opioid receptor epitopes, FPGWAEPDSNGSAGSEDAQL (SEQ ID NO:9) and GDLSDPCGNRTNLGGRDSL (SEQ ID No:11), respectively. The serum antibody titer and antibody subtypes produced in rabbits injected with the two compositions containing the different immunogens were compared.

i. Peptide conjugates. In one instance the epitopes were conjugated to KLH via a lysine residue added synthetically to the N-terminus of the epitope along with an alanine residue which acted as a spacer. In this experiment, glutaldehyde was used to effect conjugation. In the another case, the epitopes were linked to the N-terminal end of the C5a agonist YSFKPMPLaR (SEQ ID NO:1) using the solid phase peptide synthetic methodologies described above in example 1.

ii. Immunization protocol for rabbits. Rabbits were immunized s.c. with 500 μg of either the epitope-KLH or the epitope-YSFKPMPLaR (epitope-SEQ ID NO:1) constructs in compete Freund's adjuvant (GIBCO, Grand Island, N.Y.). Booster injections were administered on days 30 and 60 in incomplete Freund's's adjuvant. Serum was collected starting at day 60 post-immunization.

iii. Antibody determination. The presence of rabbit IgG specific for the peptide epitopes was determined by ELISA as previously described (8). Rabbits immunized with the epitope-C5a agonist generated high titer IgG Abs specific for the opioid receptor peptide epitopes. The rabbits immunized with the opioid receptor epitopes conjugated to the carrier protein KLH also produced high titer antibodies specific epitopes to which they were injected. These results demonstrate that the decapeptide C5a-agonist was as effective as the large molecular weight protein, KLH, conjugated to the epitope at inducing specific anti-peptide antibodies in non-rodent species.

### EXAMPLE 5

### Evaluation of C5a Peptide to Function as a Molecular Adjuvant and Epitope Delivery System for a Defined CTL Epitope of Hepatitis B Virus Surface Antigen

Experiments were performed in which YSFKPMPLaR (SEQ ID NO: 1) was used as a molecular adjuvant for inducing antigen-specific CTL responses against a defined CTL peptide epitope from the hepatitis B surface antigen (HBsAg). The HBsAg CTL epitope was covalently attached to either the N-terminus of YSFKPMPLaR (SEQ ID NO: 1) (C5a-active constructs) or to its C-terminus (C5a-inactive constructs). Mice were immunized with these C5a-active and C5a-inactive constructs in the absence of any added adjuvant in order to evaluate the ability of YSFKPMPLaR (SEQ ID NO: 1) to provide the necessary signals and/or targeting required for the induction of an antigen-specific CTL response. Immunizations were also performed with C5a-active constructs containing protease-sensitive linker sequences between the HBsAg CTL epitope and the C5a agonist. The results of these experiments were analyzed in terms of a possible mechanism by which YSFKPMPLaR (SEQ ID NO: 1) induces antigen-specific CTL responses and the importance of a protease-sensitive sequence between the epitope and the C5a agonist.

Materials and Methods:

Peptide synthesis. Peptides were synthesized by standard solid phase methodologies on an Applied Biosystems (Foster City, Calif.) model 430A synthesizer. Syntheses were performed on a 0.25-mmol scale and employed the Fmoc (9-fluorenylmethyloxycarbonyl) method of repetitive residue linkages. Peptide purification was accomplished with analytical and preparative HPLC on columns packed with C₁₈₋bonded silica. All peptides were characterized by amino acid compositional analysis and mass spectrometry. The details of these methods of synthesis, purification, and characterization have been described previously (Sanderson et al., J. Med. Chem. 37, 3171, 1994).

Pharmacologic assays. C5a-like agonistic activity was assessed by the ability of the peptides to induce smooth muscle contraction of human umbilical artery and i release from human PMNs according to previously published methods (Sanderson et al., 1994, supra; Sanderson et al., J. Med. Chem. 38, 3669, 1995; Finch et al., J. Med. Chem. 40, 877, 1997). Full concentration-response curves were generated for individual peptides and natural C5a in each assay and the EC₅₀ values (the concentration of peptide producing 50% of the maximal response to each peptide) were calculated. pD₂ transforms [−log EC₅₀ (M)] were calculated for each concentration-response curve and reported as the mean±SE. Peptide binding affinity to the C5aR was evaluated on intact human PMNs by a competition assay using ¹²⁵I-C5a according to previously described methods (Sanderson et al., 1995, supra; Finch et al., 1997, supra). Statistical analysis of the values obtained from pharmacologic assays was performed using one-way analysis of variance (ANOVA).

Animals. Female BALB/c (H-2^{d}) mice 10 to 12 weeks old were purchased from the Jackson Laboratory (Bar Harbor, Me.). On arrival, mice were housed in isolator cages, provided autoclaved food and water ad libitum, and quarantined for seven days. On release from quarantine, the mice were entered into the study following written protocols on file with the Animal Care and Use Committee and in compliance with the Animal Welfare Regulations (9CFR).

Immunization protocols. Groups of six to eight BALB/c mice were given bilateral s.c. injections in the inguinal area with (100 μl each side) of a PBS solution containing 50-100 μg of the peptides. Booster injections were given s.c. in the inguinal region at 21-day intervals. Sera for Ab analysis were obtained by retro-orbital bleeds of mice under CO₂ narcosis.

Spleen cell cultures and in vitro stimulation. Three weeks after secondary (2°) immunization and two weeks after tertiary (3°) immunization, two to three mice from each experimental group were sacrificed by cervical dislocation and their spleens removed aseptically. Pooled splenic single-cell suspensions were prepared in RPMI-1640 medium (Sigma, St. Louis, Mo.) containing 10% FBS (Hyclone, Salt Lake City, Utah) and the following supplements: 10 mM HEPES buffer, 50 μM 2-ME, 2 mM L-glutamine, 50 μg/ml gentamicin sulfate, 100 U/ml penicillin, and 50 μg/ml streptomycin (all supplements from Sigma). This supplemented complete medium is designated RP10-SC. For culture, 75×10⁶ pooled spleen cells in 5 ml of RP10-SC were pipetted into a 25 cm² T-flask (Corning). Next, 5 ml of RP10-SC containing 150 nM of synthetic, L^{d} MHC class I-restricted peptide, IPQSLDSWWTSL (SEQ ID NO: 12) were added to the flask. The flasks were incubated undisturbed in an upright position at 37° C. in 5% CO₂. After 4 days of incubation, the cells were recovered from the T-flasks, washed, once by centrifugation in fresh RP10-SC, resuspended in 5 ml RP10-SC, counted, and adjusted to 5×10⁶ viable cells/ml.

Target cell lines. The specific cell target used for measuring CTL activity was P815S, a transfectant cell line of P815 (H-2^{d}) expressing the HBsAg (14). P815S was grown in RP10-SC medium containing 400 μg/ml geneticin disulfate (G418, Sigma). The parental H-2^{d} mastocytoma cell line, P815 (ATTC #TIB64) grown in RP10-SC medium, was used as a non-specific target for CTL assays to measure % non-specific lysis. In all the experiments shown, this value was less than 5% at effector-to-target ratios of 50:1.

Target cell labeling. The target cells, either P815S or P815, were washed 2 times in RP10-SC. For labeling, 5×10⁶ target cells were mixed with 250 μCi ⁵¹Cr-sodium chromate [400-1200 Ci (14.8-44.4 TBq)]/g; NEN Dupont, Boston, Mass.) in a 1.0 ml volume in a 50 ml conical tube and incubated in a 37° C. water bath for 90 minutes. After incubation, the labeled cells were washed 3 times by centrifugation using 15 ml volumes of fresh RP10-SC and allowed to stand at room temperature for 30 minutes. The pelleted cells were resuspended in RP10-SC at 1×10⁶ cells/ml.

Cytotoxicity assay. The recovered splenic effector cells at 5×10⁶ cells/ml were serially diluted in triplicate in wells of round-bottom 96 well plates (Corning, 25850) in a total volume of 100 μl/well and using RP10-SC as the diluent. Next, 100 μl volumes of ⁵¹Cr-labeled targets, P815S or P815, at 1×10⁶ cells/ml were added to the wells. Maximum release (MR) wells contained 100 μl of target cells and 100 μl of 2% (v/v) Tween 20 while spontaneous release (SR) wells contained labeled cells in medium alone. Effector-to-target ratios of 50:1, 25:1, 12.5:1, and 6.25:1 were routinely employed. The plates were centrifuged at 400×g and incubated at 37° C. in 5% CO₂ for 4 hrs. After incubation, the supernatant fractions in the wells were collected using a Skatron Supernatant Collection System (Skatron Instruments, Sterlin, Va.). The amount of ⁵¹Cr radioactivity in the supernatant fractions was measured using a Wallac 1470 Wizard gamma counter (Turku, Finland). Percent specific lysis was calculated as [(experimental release−SR/(MR−SR)]×100. The SR was always less than 10% of the MR. All assays were performed in triplicate.

Results:

Peptide design. Peptide immunogens were designed to evaluate the requirement for C5a agonist activity in the induction of Antigen-specific CTL responses. C5a-active constructs were generated by the covalent attachment of the HBsAg CTL epitope to the N-terminus of the C5a agonist: IPQSLDSWWTSLYSFKPMPLaR (SEQ ID NO: 13) IPQSLDSWWTSLRRYSFKPMPLaR (SEQ ID NO: 14), and IPQSLDSWWTSLRVRRYSFKPMPLaR (SEQ ID NO: 15). This positioning of the CTL epitope relative to the C5a agonist leaves the biologically important conformational features expressed in the C-terminal region of YSFKPMPLaR (SEQ ID NO: 1) free to interact with C5aRs expressed on the cells involved in antigen uptake and processing. The latter two peptides were designed to evaluate if predicted protease-sensitive linker sequences placed between the HBsAg CTL epitope and the C5a agonist might facilitate intracellular release of the epitope into antigen presenting pathways and thereby enhance the response. The linkers consisted of a dibasic double-Arg (RR) sequence, which is susceptible to cleavage by proteases of the subtilisin family and trypsin-like proteases. The other was a sequence sensitive to the ubiquitous intracellular subtilisin-like protease furin, RVRR (SEQ ID NO: 19). This latter sequence is found at the junction of the A and B fragments of diphtheria toxin (DT) and it is believed that furin plays a prominent role in the intracellular proteolytic activation of DT and several other bacterial toxins as well as in the processing of proproteins and prohormones that contain the consensus sequence RX(K/R)R. C5a-inactive constructs were generated by blocking the functionally important carboxyl group on the C-terminal Arg of YSFKPMPLaR (SEQ ID NO:1) with either the HBsAg CTL epitope, YSFKPMPLaRRRIPQSLDSWWTSL (SEQ ID NO: 16) or with a Gly residue, IPQSLDSWWTSLRRYSFKPMPLaRG (SEQ ID NO: 17).

Pharmacologic activities. All peptides were evaluated for C5a agonist activities in assays that measured peptide-mediated contraction of smooth muscle in human umbilical artery (Table 3), the release of MPO from human PMNs (Table 4), and binding to C5aRs expressed on the surface of human PMNs (Table 5). Constructs in which the HBsAg CTL epitope was attached to the N-terminus of the C5a agonist, IPQSLDSWWTSLYSFKPMPLaR (SEQ ID NO: 13), IPQSLDSWWTSLRRYSFKPMPLaR (SEQ ID NO: 14), and IPQSLDSWWTSLRVRRYSFKPMPLaR (SEQ ID NO: 15) behaved as full agonists relative to natural C5a with potencies and C5aR binding affinities comparable to or greater than YSFKPMPLaR (SEQ ID NO: 1). In contrast, the construct in which the functionally important C-terminal carboxyl group of the C5a agonist moiety was blocked with the HBsAg CTL epitope YSFKPMPLaRRRIPQSLDSWWTSL (SEQ ID NO: 16) was significantly less potent in umbilical artery contraction (Table 3), inactive in MPO release from PMNs (Table 2), and bound poorly to the C5aR (Table 5) relative to both natural C5a and YSFKPMPLaR (SEQ ID NO: 1). Similarly, the construct in which a Gly residue blocked the C-terminal carboxyl group of the agonist moiety IPQSLDSWWTSLRRYSFKPMPLaRG (SEQ ID NO: 17) was significantly less potent than its C5a-active counterparts in umbilical artery contraction (Table 3) and bound with significantly less affinity to the C5aR (Table 5). This construct was unable to induce a full response relative to natural C5a in MPO release from PMNs (Table 4). CTL responses are induced in mice when the L^{d} MHC class I restricted peptide, S₂₈₋₃₉, of HBsAg is covalently attached to the N-terminus of the C5a agonist via an Arg-Arg linkage. Initial experiments were designed to evaluate CTL induction by the free L^{d} MHC class I-restricted peptide S₂₈₋₃₉, IPQSLDSWWTSL (SEQ ID NO: 12), the same peptide with two Arg residues added to the C-terminus, IPQSLDSWWTSLRR (SEQ ID NO: 18), the free C5a agonist, YSFKPMPLaR (SEQ ID NO: 1) and admixtures of the above peptides. Of particular interest was the evaluation of C5a-active constructs in which the HBsAg CTL epitope was covalently attached either directly to the N-terminus of the C5a agonist IPQSLDSWWTSLYSFKPMPLaR (SEQ ID NO: 13) or through the double-Arg, protease-sensitive linker IPQSLDSWWTSLRRYSFKPMPLaR (SEQ ID NO: 14). The administered amounts of the latter two constructs were adjusted to reflect amounts equal (by weight) to that of the free HBsAg CTL epitope based on relative molecular mass. Mice in each group received two injections, spaced 21 days apart, of the indicated construct. Mice in each group were tested for splenic CTL activity at day 42 as described in Materials and Methods. As shown in Table 6, only the group injected with the double-Arg-linked construct IPQSLDSWWTSLRRYSFKPMPLaR (SEQ ID NO: 14) exhibited a significant CTL response against the P815S transfected target cells.

C5a agonist activity is necessary for the induction of antigen-specific CTL responses. To evaluate the necessity of C5a agonist activity in the induction of antigen-specific CTL responses, mice were immunized with C5a-active and C5a-inactive HBsAg CTL epitope-containing constructs. C5a-active constructs were generated by the covalent attachment, via the protease-sensitive, double-Arg linker sequence, of the HBsAG CTL epitope to the N-terminus of the C5a agonist IPQSLDSWWTSLRRYSFKPMPLaR (SEQ ID NO: 14). C5a-inactive constructs were generated by blocking the functionally important carboxyl group on the C-terminal Arg of the C5a agonist with either the HBsAg YSFKPMPLaRRRIPQSLDSWWTSL (SEQ ID NO:16) or a Gly residue IPQSLDSWWTSLRRYSFKPMPLaRG (SEQ ID NO: 17). The double-Arg-containing HBsAg CTL epitope IPQSLDSWWTSLRR (SEQ ID NO: 18) was used as a control. As shown in FIG. 1, only mice that were immunized with the C5a-active construct IPQSLDSWWTSLRRYSFKPMPLaR (SEQ ID NO:14) generated an antigen-specific CTL response. The phenotype of the effector cells responsible for the in vitro cytolytic activity in these experiments was determined to be CD8+ as judged by the ability of a rat anti-mouse lyt 2.2 MAb (2.43, ATCC TIB-210) to almost completely inhibit (greater than 90% inhibition) the cytolytic process. In contrast, a rat anti-mouse L3T4 monoclonal antibody (GK1.5, ATCC TIB-207) known to block CD4⁺T cell activity had no effect on the in vitro cytolysis induced by the active constructs (data not shown).

CTL responses are induced only by C5a-active constructs containing a protease-sensitive linker sequence between epitope and C5a agonist. To evaluate the requirement for a protease-sensitive linkage between the HBsAg CTL epitope and the C5a agonist for CTL induction, mice were immunized with C5a-active constructs in which the HBsAg CTL epitope was covalently attached directly to the N-terminus of the C5a agonist IPQSLDSWWTSLYSFKPMPLaR (SEQ ID NO: 13) or separated by protease-sensitive linker sequences IPQSLDSWWTSLRRYSFKPMPLaR (SEQ ID NO: 14) and IPQSLDSWWTSLRVRRYSFKPMPLaR (SEQ ID NO: 15). As noted previously, the double-Arg (RR) sequence is sensitive to cleavage by proteases of the subtilisin family and other trypsin-like proteases. The RVRR (SEQ ID NO: 19) sequence is a motif recognized by the intracellular protease furin. The results shown in FIG. 2 indicate that of the three C5a-active constructs, only those containing the protease-sensitive linker sequence between the HBsAg CTL epitope and the C5a agonist were capable of inducing an antigen-specific CTL response in mice. Although the RVRR-containing construct was able to elicit a specific CTL response that was significantly above background levels, the response did not exhibit an enhanced magnitude of lysis or more rapid kinetics of induction when compared with that of the RR-containing construct (data not shown). This finding suggests that intracellular furin likely plays a less significant role, or perhaps no role, in the processing of the epitope-C5a agonist construct than other intracellular proteases with specificity for basic or dibasic residues.

Antibodies to the HBsAg CTL epitope or the C5a agonist are not produced by immunization with the HBsAg CTL epitope-C5a agonist constructs. Previous studies showed that immunization of mice in the presence of additional adjuvant with MUC1 epitope-C5a agonist and opioid receptor epitope-C5a agonist constructs (see previous examples) induced antibody responses to the MUC1 and opioid receptor epitopes and the full-length, intact proteins. Thus, it was of interest to determine whether sera from mice immunized with the control peptides and epitope-C5a agonist constructs contained antibody directed against any of the peptides. Mice were bled at various times following injection and the sera from all groups were tested by ELISA for reactivity with YSFKPMPLaR (SEQ ID NO: 1), IPQSLDSWWTSL (SEQ ID NO: 12), IPQSLDSWWTSLRR (SEQ ID NO: 18), and IPQSLDSWWTSLRRYSFKPMPLaR (SEQ ID NO: 14). These analyses failed to show binding to either the C5a agonist or the CTL epitopes (optical densities equal to normal mouse serum at a 1:50 dilution of the serum). However, sera taken from mice following three injections of IPQSLDSWWTSLRRYSFKPMPLaR (SEQ ID NO: 14) yielded an ELISA titer of 1:1600 against the immunizing peptide, but did not bind the free peptides IPQSLDSWWTSLRR (SEQ ID NO: 18) or YSFKPMPLaR (SEQ ID NO: 1). These results suggest that the C5a-active, RR-containing construct contributes to the formation of a neo-B cell epitope that is presented via the class II pathway.

TABLE 3
Immunogen Activity in Smooth Muscle Contraction
of Human Umbilical Artery

| | | EC₅₀ | |
|---|---|---|---|
| Peptide (SEQ ID NO:) | pD₂ ± SE^{a} | (μm)^{b} | n |
| C5a | 8.77 ± 0.14 | 0.002 | 3 |
| YSFKPMPLaR (1) | 6.99 ± 0.22 | 0.010 | 3 |
| IPQSLDSWWTSLYSFKPMPLaR (13) | 6.68 ± 0.17 | 0.21 | 3 |
| IPQSLDSWWTSLRRYSFKPMPLaR (14) | 6.79 ± 0.31 | 0.161 | 3 |
| YSFKPMPLaRRRIPQSLDSWWTSL (16) | 4.41 ± 0.06* | 39.4 | 3 |
| IPQSLDSWWTSLRRYSFKPMPLaRG (17) | 5.17 ± 0.13* | 6.78 | 3 |
| IPQSLDSWWTSLRVRRYSFKPMPLaR | 6.67 ± 0.22 | 0.18 | 3 |
| (15) | | | |

^{a}pD₂ = −log Ec₅₀ (M) expressed as mean ± SE
^{b}EC₅₀, concentration of peptide resulting in 50% maximum contraction
n represents the number of measurements performed
*significant change from YSFKPMPLaR, P < 0.05

TABLE 4
Immunogen Activity in MPO Release for Human PMNs

| | | EC₅₀ | |
|---|---|---|---|
| Peptide (SEQ ID NO:) | pD₂ ± SE^{a} | (μm)^{b} | n |
| C5a | 8.50 ± 0.39 | 0.003 | 3 |
| YSFKPMPLaR (1) | 5.88 ± 0.17 | 1.32 | 3 |
| IPQSLDSWWTSLYSFKPMPLaR (13) | 5.80 ± 0.04 | 1.58 | 3 |
| IPQSLDSWWTSLRRYSFKPMPLaR (14) | 6.27 ± 0.29 | 0.54 | 3 |
| YSFKPMPLaRRRIPQSLDSWWTSL (16) | >3 | >1 mM | 3 |
| IPQSLDSWWTSLRRYSFKPMPLaRG | 5.59 ± 0.28^{†} | 2.56 | 3 |
| (17) | | | |
| IPQSLDSWWTSLRVRRYSFKPMPLaR | 5.61 ± 0.19 | 2.15 | 3 |
| (15) | | | |

^{a}pD₂ = −log Ec₅₀ (M) expressed as mean ± SE
^{b}EC₅₀, concentration of peptide resulting in 50% maximum release of myeloperoxidase
n represents the number of measurements performed
^{†}This peptide displayed partial agonist activity up to 1 mM achieving 49 ± 9% of the maximum C5a-induced enzyme release (P > 0.05) compared to YSFKPMPLaR (SEQ ID NO: 1).

TABLE 5
Immunogen Binding Affinity for C5aRs on Human PMNs

| | | EC₅₀ | |
|---|---|---|---|
| Peptide (SEQ ID NO:) | pD₂ ± SE^{a} | (μm)^{b} | n |
| C5a | 9.43 ± 0.11 | 0.0004 | 3 |
| YSFKPMPLaR (1) | 5.63 ± 0.10 | 2.34 | 3 |
| IPQSLDSWWTSLYSFKPMPLaR (13) | 5.43 ± 0.16 | 3.69 | 3 |
| IPQSLDSWWTSLRRYSFKPMPLaR (14) | 6.38 ± 0.17* | 0.416 | 3 |
| YSFKPMPLaRRRIPQSLDSWWTSL (16) | 3.33 ± 0.09* | 465 | 3 |
| IPQSLDSWWTSLRRYSFKPMPLaRG | 4.77 ± 0.33* | 17.0 | 3 |
| (17) | | | |
| IPQSLDSWWTSLRVRRYSFKPMPLaR | 6.28 ± 0.14* | 0.529 | 3 |
| (15) | | | |

^{a}pD₂ = −log Ec₅₀ (M) expressed as mean ± SE
^{b}EC₅₀, concentration of peptide resulting in 50% inhibition of ¹²⁵I-C5a binding
n represents the number of measurements performed
*Significant change from YSFKPMPLaR, P < 0.05.

TABLE 6
Percent Specific Lysis of ⁵¹Cr-Labeled P815S
Target Cells from Mice Immunized with Various
HBsAg CTL Epitope/C5a Agonist Peptides.

| | % Specific Lysis^{a} | | | |
|---|---|---|---|---|
| Peptide (SEQ ID NO:) (μg | Effector: Target Ratio | | | |
| injected)^{b} | 50:1 | 25:1 | 12.5:1 | 6.25:1 |
| IPQSLDSWWTSL (12) (25) | 7 | 5 | 3 | 3 |
| IPQSLDSWWTSLYSFKPMPLaR (13) | 6 | 4 | 2 | 1 |
| (47) | | | | |
| IPQSLDSWWTSLRR (18) (25) | 2 | 2 | 1 | 0 |
| IPQSLDSWWTSLRRYSFKPMPLaR | 32 | 23 | 14 | 9 |
| (14) (42) | | | | |
| YSFKPMPLaR (1) (25) | 1 | 2 | 0 | 1 |
| IPQSLDSWWTSL (12) (25) + | 1 | 2 | 1 | 1 |
| YSFKPMPLaR (1) (25) | | | | |
| IPQSLDSWWTSLRR (18) (25) + | 2 | 0 | 0 | 0 |
| YSFKPMPLaR (1) (25) | | | | |
| Normal Spleen Cells | 1 | 1 | 1 | 0 |

^{a}The CTL response was measured against the H-2^{d} cell line (P815S) a transfectant that expresses the HBsAg. Normal P815 cells were used as a measure of non-specific lysis. % lysis against ⁵¹Cr-labeled P815 targets ranged between 0-5% at a 50:1 effector-to-target ratio (data not shown).
^{b}BALB/c mice were injected s.c. with the indicated peptides in PBS. Mice were boosted on day 21 and spleen cell suspensions from each group were prepared on day 42 and restimulated as in vitro cultures for 4 days in the presence of HBsAg S₂₈₋₃₉ peptide, IPQSLDSWWTSL.

Discussion:

As can be seen from the results presented above, the conformationally biased C5a agonist YSFKPMPLaR (SEQ ID NO: 1) serves as an effective molecular adjuvant, in this case by inducing antigen-specific CTL responses against a well-defined T cell epitope derived from the HBsAg. The CTL responses were CD8⁺ and were observed only in mice that were immunized with C5a-active constructs in which the HBsAg CTL epitope was covalently attached to the N-terminus of YSFKPMPLaR (SEQ ID NO: 1) (i.e., IPQSLDSWWTSLRRYSFKPMPLaR (SEQ ID NO: 14) and IPQSLDSWWTSLRVRRYSFKPMPLaR (SEQ ID NO: 15)). This arrangement leaves the biologically important conformational features in the C-terminal region of YSFKPMPLaR (SEQ ID NO: 1) free to interact with C5aRs expressed on the cells involved in the immune responses and underscores the necessity of C5a agonist activity in the generation of the observed CTL response. However, the presence of C5a agonist activity in the epitope-C5a agonist constructs alone was not sufficient in generating HBsAg-specific CTL responses. This was indicated by the lack of a CTL response in mice immunized with IPQSLDSWWTSLYSFKPMPLaR (SEQ ID NO: 13), despite the fact that this construct behaved as a full agonist of C5a. It is noteworthy that this C5a-active construct lacked a protease-sensitive linker sequence separating the epitope moiety from the C5a agonist moiety that was present in the two C5a-active constructs that generated a CTL response—either the double-Arg (RR) or the furin protease-specific sequence RVRR (SEQ ID NO: 19). That CTL responses were observed only in mice immunized with C5a-active constructs that contained these protease-sensitive sequences between the epitope and C5a agonist moieties supports the concept that during the internalization of the C5aR/ligand complex an intracellular cleavage event may separate the epitope from the agonist to facilitate the entry of the epitope into intracellular antigen presentation pathways. It should be noted that the failure of the free HBsAg peptide epitope to elicit a CTL response could be attributable to degradation after injection of internalization. Thus, it might be considered that the attachment of the epitope peptide to the N-terminus of the RR-containing C5a agonist peptide might, in part, reduce the sensitivity of the epitope to degradative effects. Such stabilization of the CTL epitope could, in part, contribute to the effectiveness of the co-linear RR-containing constructs. However, it is unlikely that such a phenomenon represents the sole mechanism involved since blocking the C-terminus of the C5a agonist moiety in these constructs abrogates their ability to elicit a CTL response, thereby indicating an essential role for C5a agonist activity in the observed responses.

It is also noteworthy that the C5a-active constructs IPQSLDSWWTSLRRYSFKPMPLaR (SEQ ID NO: 14) and IPQSLDSWWTSLRVRRYSFKPMPLaR (SEQ ID NO: 15) induced robust CTL activity after a 2° boost in the absence of any added adjuvant. This observation suggests that the C5a agonist moiety is capable of eliciting the T cell help necessary to induce the observed CD8⁺ CTL response. It is likely that this T cell involvement emanates from the ability of the C5a agonist moiety to induce the release of immunopotentiating cytokines from C5aR-bearing APCs with which the epitope-C5a agonist constructs interact. This supposition is supported by the fact that C5a has been shown to induce the synthesis and release of IL-1β, IL-20), IL-8, and IL-12 from human monocytee and IL-1β, IL-6, IL-8, IL-12, TNF-α, and IFN-γ from human dendritic cells. The C5a agonist moiety, therefore, appears capable of both targeting the attached epitope to C5aR-bearing APCs and eliciting the appropriate immunopotentiating activity. Thus, the YSFKPMPLaR (SEQ ID NO: 1) moiety of the constructs used in these immunizations can be viewed as a molecular entity that embodies adjuvant properties characteristic of both a “targeting vehicle” and an “immunomodulator”. Finally, mice immunized with YSFKPMPLaR (SEQ ID NO: 1) containing constructs displayed no outward physical signs that would be characteristic of a C5a-mediated anaphylactic response. This in vivo use of YSFKPMPLaR (SEQ ID NO: 1) and lack of associated toxicity in consistent with the response-selective activities that have been observed in vitro.

The results described herein are consistent with a mechanism described in the previous examples. The YSFKPMPLaR (SEQ ID NO: 1) moiety of the HBsAg constructs interacts with C5aRs expressed on the surface of APCs to induce the synthesis and release of cytokines that activate T cells. Following C5aR activation and cytokine release, the C5aR/ligand complex internalizes allowing intracellular proteases to separate the HBsAg epitope from the C5a agonist by cleaving at the double-Arg (RR) or furin-specific sequence (RVRR (SEQ ID NO: 19)) that separate these two moieties. The HBsAg epitope then associates with MHC class I determinants that are subsequently expressed on the APC surface. While this mechanism of CTL induction by the C5a agonist-containing constructs remains is but one of several possibilities, it may involve a novel pathway of exogenous MHC class I antigen presentation. Since it had been generally assumed that class I-mediated antigen presentation involved the generation of peptides from endogenously synthesized proteins, the finding that extracellular soluble proteins could be taken up by professional phagocytes (macrophages and dendritic cells), processed in the cytoplasm or perhaps endosomes to yield antigenic peptides, which are presented in association with MHC class I molecules, is of considerable significance. Presentation of extracellular antigens would be expected to be most efficient when they are particulate in nature and, consequently, are more susceptible to phagocytosis by macrophages and dendritic cells. In the case of the C5a agonist constructs, it is possible that targeting the C5aR on such cells might accomplish a similar enhancement of presentation in the MHC class I pathway. Such a proposal seems especially tenable in light of the recent findings that subunits of several bacterial toxins, especially anthrax toxin, when coupled to protein and peptide antigens, are capable of effecting internalization of the antigens and delivering them into the class I presentation pathway with resultant antigen-specific CTL production. Finally, it is noteworthy that a proteosome-independent, furin-dependent viral antigen processing pathway where cleavage occurs in the Golgi or post-Golgi secretory pathway has been recently described. Again, this finding suggests that intersection of internalized antigens/peptides with elements of the anterograde secretory pathway (endosomal or trans-Golgi region), as may occur with C5aR/ligand complexes, could result in processing events and association with unoccupied class I molecules that are in transit through this pathway.

Although an exogenous pathway of intracellular processing of the epitope-RR-C5a agonist constructs appears to be a plausible mechanism, it is also possible that the HBsAg epitope peptide is introduced onto MHC class I determinants expressed on the surface of the APC. Thus, the co-linear peptides containing the RR-C5a agonist moiety could bind to the surface of APCs and, after proteolytic cleavage of the scissile linkage, the HBsAg CTL epitope could displace lower affinity endogenous peptide in cell surface class I molecules. At present, the relative contribution of extracellular and intracellular processing events in CTL induction mediated by the C5a agonist moiety has not been assessed. Further in vitro experiments are being designed to address this issue.

In contrast to formulating peptide and/or protein antigens as particulates or as toxin subunit-conjugates or other derivatives, the C5a agonist peptide, rather than serving as an inert carrier, might provide the added benefit of delivering immunopotentiating signals. Accordingly, such constructs, containing either covalently linked peptides or proteins, might be of particular benefit in those situations where the target proteins or peptides are nominally immunogenic irrespective of the delivery vehicle or construct employed. In the case of peptides, a further advantage of this technology is that the C5a agonist constructs are relatively simple to produce and do not require recombinant technologies and associated protein purification methodologies or specialized formulation procedures.

The antigen-specific responses to well-defined T cell and B cell epitopes observed in our studies support the potential use of YSFKPMPLaR (SEQ ID NO: 1) and other response-selective C5a agonists as molecular adjuvants for inducing a defined spectrum of humoral and/or cellular responses against peptide, protein, and, possibly, non-protein antigens. Such as possibility would provide a broad-based adjuvant/delivery technology that would be applicable to a number of infectious and oncologic diseases in either prophylactic or therapeutic settings.

### EXAMPLE 6

### Evaluation of Nicotine Vaccine Compositions for Recruitment and Activation of Antigen Presenting Cells (APCs) and Stimulation of an Immune Response in Mice

i. Construction of the Nicotine Vaccine Compositions. The nicotine vaccine composition was synthesized by first generating the nicotine hapten 5 by the route shown in Scheme 1.

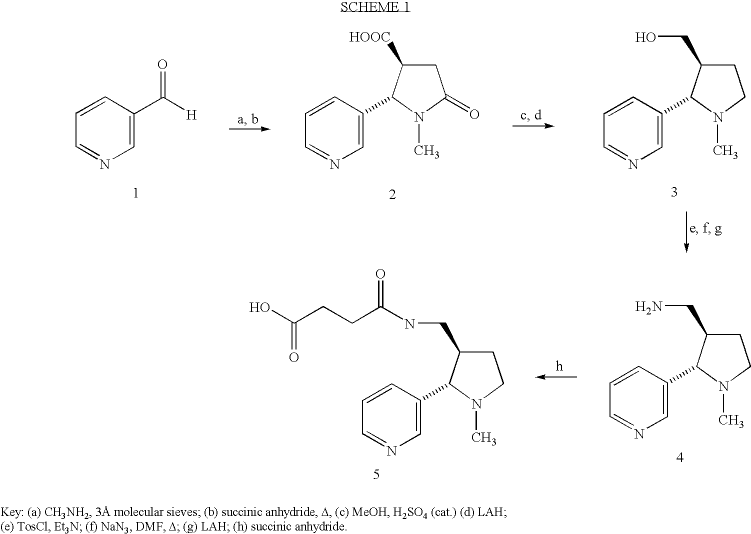

Once generated, 5 was then converted into an activated ester and coupled to the N-terminus of YSFKPMPLaR (the molecular adjuvant, SEQ ID NO:1) and YKQGGFLGLYSFKPMPLaR (a B cell epitope-molecular adjuvant construct, SEQ ID NO:2) during the solid-phase synthesis of these peptides according to Scheme 2.

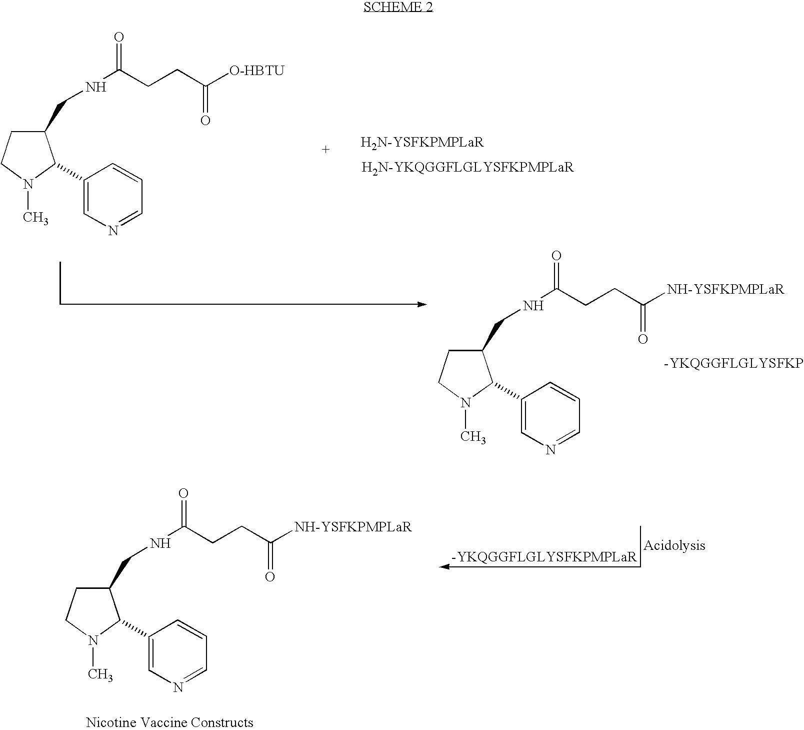

The two C5a-active nicotine vaccine constructs (Nic-YKQGGFLGLYSFKPMPLaR (Nic-SEQ ID NO:2) and Nic-YSFKPMPLaR (Nic-SEQ ID NO:1)) were purified and characterized by standard methods as detailed above.

ii. Animal Models. Five groups of male Wistar rats (ca 275 g) were immunized every other week with: 1) PBS 2) Nic-YSFKPMPLaR (Nic-SEQ ID NO:1), 3) Nic-YSFKPMPLaR (Nic-SEQ ID NO:1) in Freund's adjuvant, 4) Nic-YKQGGFLGLYSFKPMPLaR (Nic-SEQ ID NO:2), and 5) Nic-YKQGGFLGLYSFKMPMPLaR (Nic-SEQ ID NO:2) in Freund's adjuvant. Primary immunizations were accomplished by subcutaneous (s.c.) and intrapertioneal (i.p.) injection of 100 μg of the peptides as indicated above in the presence or absences of complete Freund's adjuvant. Boosting was performed in identical fashion at two-week internals with 100 μg of the peptides in the presence of absence of Freund's adjuvant. Sera were obtained by retro-orbital sinus puncture one week after the third injection. The presence of anti-nicotine antibodies in immune serum was demonstrated by standard enzyme-linked immunosorbant assay (ELISA) using a conjugate of 5 (Nic) to various proteins and their unmodified controls as the coating antigens.

iii. Results

TABLE 7
Reactivity of antiserum from rats immunized with
nicotine-modified peptide immunogens on bovine serum
albumin (BSA) unmodified or modified with nicotine.

| | Antigen Coating the Plate | | | |
|---|---|---|---|---|
| | | | Modified Minus | |
| | Raw Data | | Unmodified | |
| | (ng/ml) | | (ng/ml) | |
| Immunogen | BSA | BSA-Nic | BSA | BSA-Nic |
| PBS | 0 | 0 | 0 | 0 |
| C5a-Nic^{a} | 466 | 4,320 | 0 | 3,854 |
| C5a-Nic | 1,304 | 8,986 | 0 | 7,682 |
| Freund's | | | | |
| B-cell-C5a-Nic^{b} | 1,298 | 35,241 | 0 | 33,943 |
| B-Cell-C5a-Nic | 0 | 15,371 | 0 | 15,371 |
| Freund's | | | | |

^{a}Nic-YSFKPMPLaR (SEQ ID NO: 1)
^{b}Nic-YKQHGGFLGLYSFKPMPLaR (SEQ ID NO: 2)

As shown in Table 7, only those rats immunized with C5-a-active constructs (Nic-YSFKPMPLaR (Nic-SEQ ID NO:1), and Nic-YKQGGFLGLYSFKMPMPLaR (Nic-SEQ ID NO:2)) generated significant concentrations (ng/m;) of anti-Nic antibodies. Although good results were obtained in the presence of adjuvant (Freund's), it is noteworthy that concentrations of anti-Nic antibodies were generated in the absence of Freund's adjuvant.

As shown in FIG. 7, when these antisera were tested on other nicotine-modified peptides, Nic-YSFKPMPLaR (C5a-Nic, (SEQ ID NO:1)) and Nic-YKQGGFLGL (B-Cell-Nic, (SEQ ID NO:6)), the antibody reactivity was still highest in the serum from rats immunized with Nic-YKQGGFLGLYSFKPMPLaR (SEQ ID NO:2) in the presence of Freund's adjuvant (172,647 ng/ml). However, Nic-YSFKPMPLaR (C5a-Nic, (SEQ ID NO:1)) in the presence and absence of Freund's adjuvant also induced responses to nicotine as determined by reactivity to C5a-Nic (Nic-YSFKPMPLaR, (SEQ ID NO:1)) and the B-Cell-Nic (Nic-YKQGGFLGL, (SEQ ID NO:6)).

### EXAMPLE 7

### Attenuation of Nicotine Induced Behavioral Effects in Rats

In order to determine the ability of the nicotine vaccine compositions of the invention to attenuate behavioral effects of nicotine addiction, 16 male Sprague-Dawley rats weighing approximately 300 grams were divided into two groups (with eight rats in each group) and each group was administered a different treatment regiment. All nicotine compositions were prepared as described in Example 6. In one treatment group, eight rats were vaccinated with 1000 mg of Nic-YKQGGFLGLYSFKPMPLaR (Nic-SEQ ID No:2) (500 mg s.c. and 500 mg i.p.) dissolved in sterile saline and boosted in identical fashion every week for 2 weeks. The second group of eight was sham immunized and boosted with sterile saline. Following the last boost, the rats' 100% free-feeding weight was slowly decreased by 15% across a week (i.e., target weight). This target weight insured that the rats would seek 32% (w/v) liquid sucrose in the experimental apparatus. On days 1 to 3, rats were taught to access 4-second deliveries of liquid sucrose in the apparatus. For the following 16 days, rats in the vaccinated and non-vaccinated groups were shifted to a or nicotine) intermixed in a quasi-random fashion within two 8-session cycles that included 4 saline and 4 nicotine sessions. On saline sessions, rats were injected s.c. with saline (0.9% NaCl) and placed in the experimental apparatus for 20 minutes. Following variable delays, 8 lights were presented for 15 seconds, but no food was delivered. Saline sessions, therefore, provided a baseline with which to determine if the vaccine affected behavior independent of the presence of nicotine. On nicotine sessions, rats were injected s.c. with nicotine (0.4 mg/kg base) and placed in the apparatus for 20 minutes. The light cues were followed by 4-second access to the sucrose. Thus, nicotine plus light signaled food access; light alone (i.e., saline sessions) signaled no food.

In early sessions, food seeking, as measured by breaks in an infrared beam where sucrose is accessed (hereinafter “dipper entries”), should be high. Dipper entries, however, should decrease in saline sessions because no sucrose is being delivered. Indeed, food seeking systematically decreased across saline sessions in a pattern similar for vaccinated and non-vaccinated rats (FIG. 8, Panel A). This outcome indicates that the vaccine does not differentially affect behavior in this preparation when nicotine is not present. In contrast, there is a clear difference in food-seeking behavior in the nicotine sessions. Dipper entries remained high in non-vaccinated rats likely reflecting a “summation” of food-seeking behavior maintained by occasional sucrose delivery and the psychomotor stimulant effects of nicotine. Notably, dipper entries in vaccinated rats decreased below the levels of non-vaccinated rats. The results depicted in FIG. 8 demonstrate the ability of the nicotine vaccine composition to attenuate the centrally mediated psychomotor effects of nicotine in vaccinated rats during the first cycle of training. This interpretation is supported by an omnibus 3-way mixed factorial analysis of variance with Group (vaccinated vs. non-vaccinated) as a between-subjects factor, and Session (1 to 4) and Session Type (nicotine or saline) as within-subject factors. The main effects of Group, Session, and Session Type were all statistically significant, ps less than or equal to 0.002. Further, the Group x Session Type interaction, F(1,14)=6.31, p=0.025 and the Session x Session Type interaction, F(3,42)=5.024, p=0.005, were significant. The Session x Group and the Session x Group x Session Type interactions were not significant, p greater than or equal to 0.34. Subsequent statistical contrasts to investigate the source of the Group x Session Type interaction were limited to comparing vaccinated and non-vaccinated rats within each session type. Vaccinated rats displayed significantly less dipper entries than non-vaccinated rats on nicotine Sessions 2 and 3, ps less than 0.05, indicating that the vaccine was effective at attenuating the effects of nicotine in this animal model.

FIG. 8, Panel B shows the index of learning-elevation score for the first light (before sucrose is given). A positive elevation score indicates learning in that more dipper-entries are occurring during the 15-second light than in a comparable time just before the light occurred (light dipper entries minus pre-light dipper entries). As can be seen, by the end of the first cycle rats are just learning that sucrose is being delivered during the light only on nicotine sessions. This index does not differ between vaccinated and non-vaccinated rats. Analyses confirm these observations. There was a main effect of Session and Session Type, p less than or equal to 0.012. No other factors were significant, p greater than or equal to 0.076. Notably, the vaccine did not produce a general insensitivity to sucrose reward. Evidence for this comes from the similar acquisition rate of anticipatory dipper entries in vaccinated and non-vaccinated rats.

As training continued and rats learned to discriminate nicotine sessions from saline sessions (FIG. 8, Panel B), the effect of the vaccine on total number of dipper entries in the nicotine sessions was lost (FIG. 8, Panel A). For the total number of dipper entries only the main effect of Session Type was significant, p<0.001. For the elevation score only the main effect of Session Type and the Session Type x Session interaction were significant, ps greater than or equal to 0.032. Overall, these data indicate that dipper entries (food seeking) are coming under the control of the nicotine cue and that the vaccine, albeit effective, does not prevent enough of the nicotine from acting as an interoceptive cue.

Six rats from each of the above treatment groups (i.e., nicotine exposed-vaccinated and nicotine exposed-non-vaccinated) were bled and serum obtained for studies on antibody titer and specificity. The titer was determined by assessing the reactivity (optical density or “OD”) of each antiserum on bovine serum albumin (BSA) and BSA modified with the nicotine hapten (BSA-Nicotine). The OD of the antiserum on BSA was subtracted from its OD on BSA-Nicotine, and the last difference in OD that resulted in greater than a 0.100 reading was considered the final titer for that antiserum. As shown in FIG. 9, the antibody titers of the six rats in the vaccinated group averaged 1:223. In contrast, the non-vaccinated group of rats had antibody titers of less than 1:50, as this was the lowest dilution utilized in these studies and none of the rats were above this dilution. Thus, a significant increase in the antibody titers of rats immunized with Nic-YKQGGFLGLYSFKPMPLaR (Nic-SEQ ID No:2) was determined.

Specificity of the vaccinated rat antiserum was addressed using a competitive ELISA. Briefly, a dilution for each rat was determined on the BSA-nicotine that resulted in 50% maximum (0.800) OD after 30 minutes. To this dilution, different concentrations of nicotine or phosphate buffered saline (i.e., no inhibitor), were added to the antiserum and allowed to incubate overnight at 4° C. The next day, these samples were added to ELISA plates coated with BSA-nicotine and the reactivity of the antiserum plus inhibitor was determined. As shown in FIG. 10, nicotine began to inhibit the antibody response between 250 and 500 pM, and reached maximum inhibition between 8,000 and 10,000 pM. These data demonstrate that the antibodies are specific for nicotine.

Collectively, these results show not only that the nicotine vaccine (Nic-YKQGGFLGLYSFKPMPLaR (Nic-SEQ ID No:2)) is capable of generating anti-nicotine antibodies in rats, but is also capable of attenuating nicotine-induced behavioral effects in the presence of high concentrations of nicotine (0.4 mg/kg). Moreover, the immune response retains its resiliency over the course of approximately 2 months time. Most remarkable is that fact that these in vitro and in vivo immune responses were generated in the complete absence of any added adjuvant. Thus, this vaccine design generates immune responses with the desired nicotine specificity and longevity even in the presence of large amounts of nicotine.

While certain preferred embodiments of the present invention have been described and specifically exemplified above, it is not intended that the invention be limited to such embodiments. Various modifications may be made to the invention without departing from the scope and spirit thereof as set forth in the following claims.

### REFERENCES

1. Rammensee et al. (1993) “Peptides Naturally Presented by MHC Class I Molecules,” Ann. Rev. 1 mm. 11:213-244.
2. Ausubel et al., “Current Protocols in Molecular Biology,” John Wiley & Sons, Inc., 1995.
3. Barclay, et al., (1993) The Leucocyte Antigen Facts Book. Academic Press, Harcourt Brace and Co., London.
4. Ember, J. A., Sanderson, S. D., Taylor, S. M., Sawahara, M., and Hugli, T. I. (1992) “Biological activity of synthetic analogues of C5a anaphylatoxin”. J. Immunol. 148: 3165-3173.
5. Robert R Buchner, Shawn M. Vogen, Wolfgang Fischer., Marilyn L. Thoman, Sam D. Sanderson, and Edward L. Morgan. (1996) “Anti-Human kappa opioid receptor antibodies characterization of site-directed neutralizing antibodies specific for a peptide κR(33-52) derived from the predicted amino-terminal region of the human kappa receptor,” J. Immunol. (In press).
6. Morgan, E. L. (1996) “Regulation of human B lymphocyte activation by opioid peptide hormones. Inhibition of IgG production by opioid receptor class (μ-, κ-, and, δ-) selective agonists”, J. Neuroimmunol. 65:21.
7. Sanderson, S. D., L Kirnarsky, S. A. Sherman, J. A. Ember, A. M. Finch, and S. M. Taylor. (1994) “Decapeptide agonists of human C5a: the relationship between conformation and spasmogenic and platelet aggregatory responses”, J. Med. Chem. 38: 3171-3180.
8. Morgan, E. L., J. A. Ember, S. D. Sanderson, W. Scholz, —R. Buchner, RD. Ye, T. E. Hugli (1993) “Anti-C5a receptor antibodies. I. Characterization of neutralizing antibodies specific for the human C5a receptor”. J. Immunol. 151: 377.
9. Hobbs, M. V., R. A. Houghten, J. A. Janda, W. O. Weigle, and E. L. Morgan, E. L. (1989) “Induction of human B cell differentiation by Fc region activators. I. Identification of an active tetrapeptide”, Clinical Immunol. Immunopathol. 50:251.

1. A compound comprising a molecular adjuvant linked to a nicotine hapten, the molecular adjuvant comprising a targeting ligand having binding affinity for a characteristic determinant of an antigen presenting cell, the targeting ligand being functionally linked to the nicotine hapten, whereby binding of the molecular adjuvant to the antigen presenting cell determinant activates the antigen presenting cell, effecting delivery of the nicotine hapten to an antigen presenting pathway of the antigen presenting cell.

2. The compound of claim 1 wherein the targeting ligand of the molecular adjuvant binds specifically to a determinant comprising an immunomodulatory receptor of the antigen presenting cell.

3. The compound of claim 1 wherein the targeting ligand of the molecular adjuvant binds specifically to a receptor selected from the group consisting of C5a receptor, IFN-gamma receptor, CD21 (C3d) receptor, CD64 (FcgRI) receptor, and CD23 (FceRII) receptor.

4. The compound of claim 1 wherein the targeting ligand of the molecular adjuvant binds specifically to a C5a receptor and is selected from the group consisting of C5a and a peptide agonist analog of C5a comprising the C-terminal ten residues of C5a.

5. The compound of claim 1 wherein the targeting ligand of the molecular adjuvant is a peptide comprising the sequence YSFKKPMPLaR, which is SEQ ID NO:1.

6. The compound of claim 1 wherein the targeting ligand of the molecular adjuvant is a peptide comprising the sequence YKQGGFLGLYSFKPMPLaR, which is SEQ ID NO:2.

7. The compound of claim 1 wherein the nicotine hapten has a structure corresponding to: 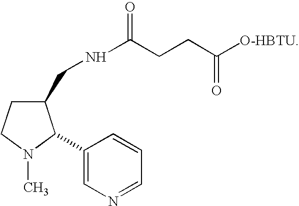
.

8. The compound of claim 1 wherein the nicotine hapten has a structure selected from the group consisting of:

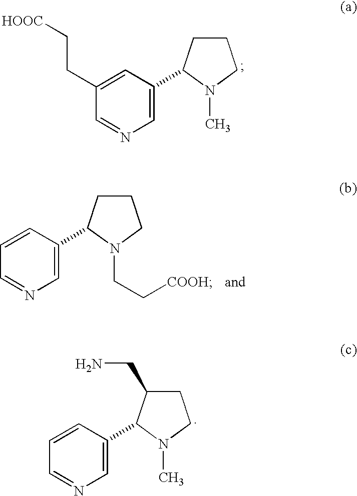

9. The compound of claim 1 wherein the nicotine hapten is linked to the molecular adjuvant by a spacer moiety.

10. The compound of claim 4 wherein the nicotine hapten is linked to the N-terminus of the peptide comprising the molecular adjuvant.

11. The compound of claim 10 wherein the nicotine hapten is linked to the N-terminus of the peptide comprising the molecular adjuvant by an amide bond.

12. The compound of claim 1 wherein the compound has a structure corresponding to:

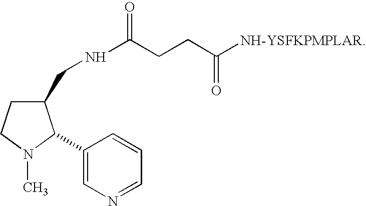

13. The compound of claim 1 wherein the compound has a structure corresponding to: 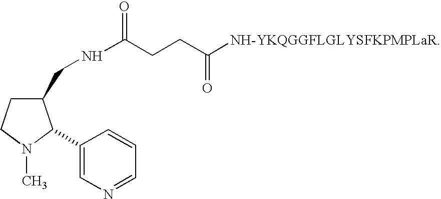
.

14. A vaccine composition comprising the compound of claim 1.

15. A vaccine composition comprising the compound of claim 4.

16. A vaccine composition comprising the compound of claim 12.

17. A vaccine composition comprising the compound of claim 13.

18. A method of treating nicotine addiction in a patient in need of such treatment, the method comprising administering to the patient a therapeutically effective amount of the compound of claim 1.

19. A method of treating nicotine addiction in a patient in need of such treatment, the method comprising administering to the patient a therapeutically effective amount of the compound of claim 4.

20. A method of treating nicotine addiction in a patient in need of such treatment, the method comprising administering to the patient a therapeutically effective amount of the compound of claim 12.

21. A method of treating nicotine addiction in a patient in need of such treatment, the method comprising administering to the patient a therapeutically effective amount of the compound of claim 13.

22. A method of preventing nicotine addiction in a patient in need of such treatment, the method comprising administering to the patient a therapeutically effective amount of the compound of claim 1.

23. A method of treating nicotine addiction in a patient in need of such treatment, the method comprising administering to the patient a therapeutically effective amount of the compound of claim 4.

24. A method of treating nicotine addiction in a patient in need of such treatment, the method comprising administering to the patient a therapeutically effective amount of the compound of claim 12.

25. A method of treating nicotine addiction in a patient in need of such treatment, the method comprising administering to the patient a therapeutically effective amount of the compound of claim 13.

26. A method of producing antibodies to a nicotine hapten, the method comprising:
(a) immunizing an animal with an immunogenic effective amount of the compound of claim 1;
(b) isolating antibodies from the sera of the animal; and
(c) recovering the isolated antibodies.

27. The method of claim 26 wherein the targeting ligand of the molecular adjuvant of said compound binds specifically to a C5a receptor and is selected from the group consisting of C5a and a peptide agonist analog of C5a comprising the C-terminal ten residues of C5a.

28. The method of claim 26 wherein said compound has a structure corresponding to: 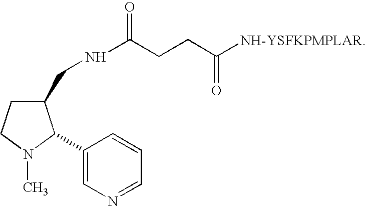
.

29. The method of claim 26 wherein said compound has a structure corresponding to: 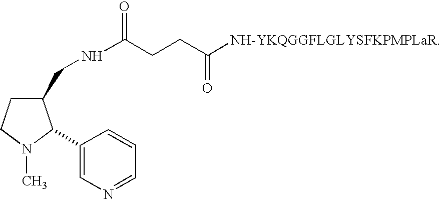
.
